(19) United States
(12) Patent Application Publication
(10) Pub. No.: US 2026/0285869 A1
(43) Pub. Date: Sep. 24, 2026

(54) COMPOUNDS, COMPOSITIONS AND METHODS THEREOF

(71) Applicant: HEPAITECH (BEIJING) BIOPHARMA TECHNOLOGY CO., LTD., Beijing (CN)

(72) Inventors: Ruichao Richard Shen, Belmont, MA (US); Xiaoguang Lei, Beijing (CN); Xiaoming Wang, Beijing (CN); Yulong Li, Beijing (CN)

(21) Appl. No.: 19/475,678

(22) PCT Filed: Apr. 18, 2024
(86) PCT No.: PCT/CN24/88496

§ 371 Date: Oct. 15, 2025

(30) Foreign Application Priority Data
Apr. 19, 2023 (WO) PCT/CN2023/089093

(51) Int. Cl. C07D 487/04; A61K 31/404; A61K 31/416; A61K 31/4184; A61K 31/519; C07D 209/08; C07D 231/56; C07D 235/06; C07D 403/04

(52) U.S. Cl. CPC C07D 487/04; A61K 31/404; A61K 31/416; A61K 31/4184; A61K 31/519; C07D 209/08; C07D 231/56; C07D 235/06; C07D 403/04

(57) ABSTRACT

Among other things, the present disclosure provides compounds, e.g., of formula I or salts thereof. In some embodiments, the present disclosure provides methods for modulating MRGPRX4 activity. In some embodiments, the present disclosure provides methods for preventing or treating conditions, disorders or diseases, e.g., MRGPRX4-associated conditions, disorders or diseases.

### CROSS-REFERENCE TO RELATED APPLICATIONS

This application claims priority to PCT Application No. PCT/CN2023/089093, filed on Apr. 19, 2023, the entirety of which is incorporated herein by reference.

### TECHNICAL FIELD

In some embodiments, the present disclosure provides compounds and compositions, among other things, useful for modulating MRGPRX4 activities. In some embodiments, provided compounds and compositions are useful for preventing or treating MRGPRX4-associated conditions, disorders or diseases, e.g., pruritus. In some embodiments, the present disclosure provides technologies for preparing provided compounds and compositions.

### BACKGROUND

Compounds are useful for many purposes including modulating biological functions and activities. It has been reported that Mas-related G-protein coupled receptor X4 (MRGPRX4) has a number of biological functions and can be associated with various conditions, disorders or diseases. For example, patients with liver condition, disorder or disease such as primary biliary cholangitis (PBC), primary sclerosing cholangitis (PSC), or progressive familial intrahepatic cholestasis (PFIC), often suffer from refractory pruritus, which severely affects quality of life and potentially leads to lassitude, fatigue, depression, and even suicidal thoughts. Recently MRGPRX4 and its agonism by bile acid and derivatives thereof have been reported to be likely associated with pruritus occurred in these diseases (e.g., Meixiong et al. MRGPRX4 is a G protein-coupled receptor activated by bile acids that may contribute to cholestatic pruritus, PNAS, 2019, 116 (21), 10525-10530; Yu et al. MRGPRX4 is bile acid receptor for human cholestatic itch, eLife, 2019, 8, e48431).

MRGPRX4 has been reported to express in at least human dorsal root ganglion (hDRG) neurons and to co-expresses with itch receptor HRH1. It has been reported that bile acids or MRGPRX4 specific agonists in the skin can activate MRGPRX4 in itch-related primary fibers, elicits Ca²⁺ responses, and induce itch in human subjects.

Small compounds that can allegedly act as MRGPRX4 modulators have been disclosed in, e.g., WO 2020/198537, US 2021/0032213, WO 2021/211839, US 2021/0340106, WO 2022/061008, etc.

### SUMMARY

In some embodiments, the present disclosure provides various compounds, e.g., compounds having the structure of formula I or salts thereof, and compositions and methods thereof. In some embodiments, provided compounds are useful as MRGPRX4 modulators. In some embodiments, provided technologies (e.g., compounds, compositions, methods, etc.) are useful for preventing or treating various conditions, disorders or diseases. In some embodiments, a condition, disorder or disease is a MRGPRX4-associated condition, disorder or disease. In some embodiments, a condition, disorder or disease is or comprises itch. In some embodiments, a condition, disorder or disease is or comprises pruritus. In some embodiments, a condition, disorder or disease is associated with administration of another therapeutic agent, e.g., a FXR agonist, a bile acid or an analog or derivative thereof, etc.

In some embodiments, the present disclosure provides a compound, wherein the compound has the structure of formula I

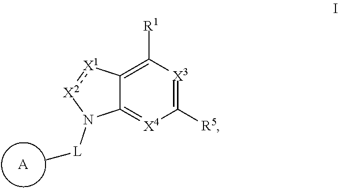

or a salt thereof, wherein:
R¹ is an isostere of —C(O)OH, or —C(O)OR¹¹, —C(O)N(R¹²)SO₂R¹³, —C(O)NR¹⁴R¹⁵, or

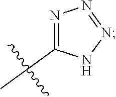 [SMILES: *c1nnn[nH]1]

—X¹═X²— is —X¹═X²— and X¹ and X² is independently —N═, —CR²—, or optionally substituted —CH═; or —X¹ ═X²— is optionally substituted —(CH₂)₂;
R² is optionally substituted C₁₋₆ alkyl;
X³ is —N═, or —CR³═;
X⁴ is —N═, or —CR⁴═;
each of R³, R⁴ and R⁵ is independently R′, OR′, halogen, CN, —NO₂, or —N(R′)₂;
each of R¹¹, R¹², R¹³, R¹⁴ and R¹⁵ is independently R′;
L is optionally substituted —(CH₂)ₘ—;
m is 1 or 2;
Ring A is an optionally substituted ring selected from a 6-10 membered aryl ring and a 5-10 membered heteroaryl ring having 1-6 heteroatoms, or Ring A is

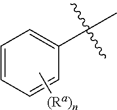

wherein each R^{a} is independently —R′, OR′, halogen, —CN, —NO₂, or —N(R′)₂ and n is 1-5;
each R′ is independently R, —OR, —C(O)R, —C(O)OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

In some embodiments, the present disclosure provides a pharmaceutical composition of a provided compounds. In some embodiments, the present disclosure provides a pharmaceutical composition comprising a provided compound or a pharmaceutically acceptable salt thereof, and a pharmaceutically acceptable carrier. In some embodiments, the present disclosure provides a pharmaceutical composition delivering a provided compound or a pharmaceutically acceptable salt thereof, and a pharmaceutically acceptable carrier.

Provided technologies are useful for many purposes. For example, in some embodiments, the present disclosure provides a method for preventing a condition, disorder or disease, comprising administering or delivering to a subject susceptible thereto an effective amount a provided compound. For example, in some embodiments, the present disclosure provides a method for treating a condition, disorder or disease, comprising administering or delivering to a subject suffering therefrom an effective amount a provided compound. In some embodiments, a condition, disorder or disease is a MRGPRX4-associated condition, disorder or disease. In some embodiments, a condition, disorder or disease is associated with MRGPRX4 activation. In some embodiments, a condition, disorder or disease is associated with MRGPRX4 activation by an agent. In some embodiments, a condition, disorder or disease is associated with MRGPRX4 interaction with an agent. In some embodiments, a condition, disorder or disease is associated with administration of an agent. In some embodiments, a condition, disorder or disease is associated with delivery of an agent. In some embodiments, an agent is a therapeutic agent. In some embodiments, an agent can bind to MRGPRX4. In some embodiments, an agent can activate MRGPRX4. In some embodiments, an agent is a FXR agonist. In some embodiments, an agent is a bile acid or an analog or derivative thereof. In some embodiments, a condition, disorder or disease is pruritus.

In some embodiments, the present disclosure provides a method for preventing a condition, disorder or disease, comprising administering or delivering to a subject susceptible thereto an effective amount a provided compound and another agent. In some embodiments, the present disclosure provides a method for treating a condition, disorder or disease, comprising administering or delivering to a subject suffering therefrom an effective amount a provided compound and another agent. For example, in some embodiments, a condition, disorder or disease is associated with Farnesoid X receptor (FXR), and an another agent is a FXR agonist. In some embodiments, a condition, disorder or disease is associated with TGR5, and an another agent is a TGR5 agonist. In some embodiments, an another agent can activate MRGPRX4. In some embodiments, an another agent is a FXR agonist. In some embodiments, an another agent is a bile acid or an analog or derivative thereof. In some embodiments, a condition, disorder or disease is a liver condition, disorder or disease. For example, in some embodiments, a condition, disorder or disease is nonalcoholic steatohepatitis (NASH).

In some embodiments, the present disclosure provides technologies, e.g., methods, reagents, etc., for preparing a compound of Formula (I) or a pharmaceutical acceptable salt thereof.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise clear from context, isomers of compounds are included. As those skilled in the art, compounds may be provided, administered, or delivered in various forms, e.g., salts (e.g., pharmaceutically acceptable salts), solvates, hydrates, esters, prodrugs, tautomers, etc.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of amino acids, in some embodiments, a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or +NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Leaving Group/LG: As described herein, a leaving group or a LG is an atom or group of atoms that detaches from the main or residual part of a substrate during a reaction or elementary step of a reaction. In some embodiments, LG is a halogen. In some embodiments, LG is —Cl. In some embodiments, LG is —OH.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents include halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —) (CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘})₂; —N(R^{∘})C(S)NR^{∘}₂; —(CH₂)₀₋₄N(R^{∘})C(O)OR^{∘}); —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR, —SC(S)SR^{∘}; —(CH₂)₀₋₄SC(O)R^{∘}; —(CH₂)₀₋₄C(O)NR^{∘}2; —C(S)NR^{∘}₂; —C(S)SR^{∘}; SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —) N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —OSiR^{∘}₃; —(C₁₋₄ straight or branched) alkylene)O—N(R^{∘})₂; or —(C₁₋₄ straight or branched) alkylene) C(O)O—N(R^{∘})₂, wherein each R^{∘} may be substituted as defined below and is independently hydrogen, C₁₋₂₀ aliphatic (e.g., C₁₋₉, C₁₋₆, C₁₋₅, C₁₋₄, etc.), C₁₋₂₀ (e.g., C₁₋₉, C₁₋₆, C₁₋₅, C₁₋₄, etc.) heteroaliphatic having 1-5 heteroatoms independently selected from nitrogen, oxygen, sulfur, silicon and phosphorus, —CH₂—(C₆₋₁₄ (e.g., C₆₋₁₀, C₆, etc.) aryl), —O(CH₂)₀₋₁(C₆₋₁₄ (e.g., C₆₋₁₀, C₆, etc.) aryl), —CH₂-(5-14 (e.g., 5-10, 5-6, 5, 6, 9, 10, 14, etc.) membered heteroaryl ring), a 3-20 (e.g., 3-10, 3-9, 3-7, 3-6, 5-10, 5-6, etc.) membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 3-20 (e.g., 3-10, 3-9, 3-7, 3-6, 5-10, 5-6, etc.) membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{∘}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene) C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3-6 (e.g., 4-6, 5-6, etc.) membered saturated, partially unsaturated, or aryl ring having 0-4 heteroatoms independently selected from nitrogen, oxygen, and sulfur. Suitable divalent substituents on a saturated carbon atom of R^{●} include ═O and ═S.

Suitable divalent substituents include the following: ═O, ═S, ═NNR*₂, ═NNHC(O)R*, ═NNHC(O)OR*, ═NNHS(O)₂R*, —NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 3-6 (e.g., 4-6, 5-6, etc.)-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 3-6 (e.g., 4-6, 5-6, etc.)-membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O (haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6 (e.g., 4-6, 5-6, etc.)-membered saturated, partially unsaturated, or aryl ring having 0-4 heteroatoms independently selected from nitrogen, oxygen, and sulfur.

In some embodiments, suitable substituents on a substitutable nitrogen include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6 (e.g., 4-6, 5-6, etc.)-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12 (e.g., 3-10, 3-7, 3-6, 5-10, 5-7, 5-6, etc.)-membered saturated, partially unsaturated, or aryl mono- or bicyclic ring having 0-4 heteroatoms independently selected from nitrogen, oxygen, and sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6 (e.g., 4-6, 5-6, etc.)-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises two or more acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9 (10,10-dioxo-10,10,10,10-tetrahydrothioxanthyl)]methyl carbamate (DBD-Tmoc), 4 methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, trichloroacetamide, chloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4 pyridone, N-methylamine, N-allylamine, N-[2-(trimethylsilyl) ethoxy]methylamine (SEM), N-3-acetoxypropylamine, N-(1-isopropyl 4 nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9 phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten) carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro 4 methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6 trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′ dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3 phenylpropionate, 4 oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6 trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4 dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl(trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl) xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

As those skilled in the art will appreciate, methods and compositions described herein relating to provided compounds generally also apply to pharmaceutically acceptable salts of such compounds.

### CERTAIN EMBODIMENTS OF COMPOUNDS

Among other things, the present disclosure provides compounds that are useful for various purposes. In some embodiments, a provided compound has the structure of formula I:

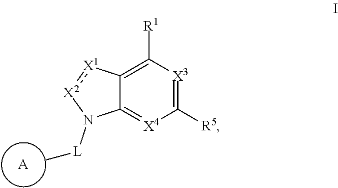

or a salt thereof, wherein each variable is independently as described herein.

Certain embodiments for various variables in various formulae (e.g., formula I and formulae in various schemes) are described herein as examples. Those skilled in the art reading the present disclosure will be able to select an embodiment for each variable and combine them; such combinations are within the scope the present disclosure. Those skilled in the art also appreciate that embodiments described for one variable (e.g., R) may be utilized for other variables that can be such variable (e.g., R′, R³, R⁴, R⁵, R¹¹, R¹², R¹³, R¹⁴, R¹⁵, etc. that can be R).

### R¹

In some embodiments, R¹ is —C(O)OR¹¹, —C(O)N(R¹²)SO₂R¹³, —C(O)NR¹⁴R¹⁵, or

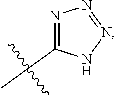 [SMILES: *c1nnn[nH]1]

wherein each variable is independently as described herein.

In some embodiments, R¹ is —C(O)OH. In some embodiments, R′ is —C(O)OR¹¹, wherein each variable is independently as described herein. In some embodiments, R¹ is —C(O)N(R¹²)SO₂R¹³, wherein each variable is independently as described herein. In some embodiments, R¹ is —C(O)NR¹⁴R¹⁵, wherein each variable is independently as described herein. In some embodiments, R¹ is

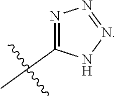 [SMILES: *c1nnn[nH]1]

In some embodiments, R¹ is —C(O)OR¹¹. In some embodiments, R¹¹ is R as described herein. In some embodiments, R¹¹ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹¹ is C₁₋₆ aliphatic. In some embodiments, R¹¹ is C₁₋₆ alkyl. In some embodiments, R¹¹ is methyl. In some embodiments, R¹¹ is ethyl.

In some embodiments, R¹ is —C(O)N(R¹²)SO₂R¹³ wherein each of R¹² and R¹³ is independently as described herein. In some embodiments, R¹ is —C(O)NHSO₂R¹³ wherein R¹³ is as described herein.

In some embodiments, R¹² is R′ described herein. In some embodiments, R¹² is R as described herein. In some embodiments, R¹² is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹² is H. In some embodiments, R¹² is optionally substituted C₁₋₆ aliphatic. In some embodiments, R¹² is C₁₋₆ alkyl. In some embodiments, R¹² is methyl. In some embodiments, R¹² is ethyl.

In some embodiments, R¹³ is R′ described herein. In some embodiments, R¹³ is R as described herein. In some embodiments, R¹³ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹³ is H. In some embodiments, R¹³ is optionally substituted C₁₋₆ aliphatic. In some embodiments, R¹³ is C₁₋₆ alkyl. In some embodiments, R¹³ is methyl. In some embodiments, R¹³ is ethyl.

In some embodiments, each of R¹² and R¹³ is independently R′ as described herein. In some embodiments, each of R¹² and R¹³ is independently R as described herein.

In some embodiments, R¹ is —C(O)NR¹⁴R¹⁵ wherein each of R¹⁴ and R¹⁵ is independently as described herein. In some embodiments, R¹ is —C(O)NHR¹⁵ wherein R¹⁵ is as described herein.

In some embodiments, R¹⁴ is R′ described herein. In some embodiments, R¹⁴ is R as described herein. In some embodiments, R¹⁴ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹⁴ is H. In some embodiments, R¹⁴ is optionally substituted C₁₋₆ aliphatic. In some embodiments, R¹⁴ is C₁₋₆ alkyl. In some embodiments, R¹⁴ is methyl. In some embodiments, R¹⁴ is ethyl.

In some embodiments, R¹⁵ is R′ described herein. In some embodiments, R¹⁵ is R as described herein. In some embodiments, R¹⁵ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R¹⁵ is H. In some embodiments, R¹⁵ is optionally substituted C₁₋₆ aliphatic. In some embodiments, R¹⁵ is C₁₋₆ alkyl. In some embodiments, R¹⁵ is methyl. In some embodiments, R¹⁵ is ethyl.

In some embodiments, each of R¹⁴ and R¹⁵ is independently R′ as described herein. In some embodiments, each of R¹⁴ and R¹⁵ is independently R as described herein.

In some embodiments, R¹ is —C(O)NR¹⁴R¹⁵, wherein each of R¹⁴ and R¹⁵ is independently H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic.

In some embodiments, R′ is —C(O)NR¹⁴R¹⁵, wherein each of R¹⁴ and R¹⁵ is independently H or optionally substituted C₁-C₆ alkyl. In some embodiments, R′ is —C(O)NR¹⁴R¹⁵, wherein each of R¹⁴ and R¹⁵ is independently H. In some embodiments, R¹ is —C(O)NR¹⁴R¹⁵, wherein R¹⁴ is H and R¹⁵ is optionally substituted C₁-C₆ alkyl. In some embodiments, R′ is —C(O)NR¹⁴R¹⁵, wherein R¹⁴ is H and R¹⁵ is methyl. In some embodiments, R′ is R′ is —C(O)NR¹⁴R¹⁵, wherein each of R¹⁴ and R¹⁵ is independently C₁-C₆ alkyl. In some embodiments, R¹ is R¹ is —C(O)NR¹⁴R¹⁵, wherein each of R¹⁴ and R¹⁵ is methyl.

In some embodiments, R¹ is an isostere of —C(O)OH. In some embodiments, a compound is a carboxylic acid isostere of a carboxylic acid compound, e.g., where R¹ is —C(O)OH. In some embodiments, a carboxylic acid isostere is one described in WO 2020/198537, US 2021/0032213, WO 2021/211839, or WO 2022/061008, the carboxylic acid isosteres of each of which are independently incorporated herein by reference.

In some embodiments, a carboxylic acid isostere is described in Ballatore et al., ChemMedChem. 2013 March; 8 (3): 385-395, the carboxylic acid isosteres of which are incorporated herein by reference.

X¹ and X²

In some embodiments, —X¹═X²— is —X¹═X²— wherein each variable is independently as described herein. In some embodiments, X¹ is independently —N═, —CR²═, or optionally substituted —CH═. In some embodiments, X¹ is —N═. In some embodiments, X¹ is optionally substituted —CH═. In some embodiments, X¹ is —CH═. In some embodiments, X¹ is —CR²═.

In some embodiments, X² is independently —N═, —CR²═, or optionally substituted —CH═. In some embodiments, X² is —N═. In some embodiments, X² is optionally substituted —CH═. In some embodiments, X² is —CH═. In some embodiments, X² is —CR²═.

In some embodiments, R² is optionally substituted C₁₋₆ alkyl. In some embodiments, R² is C₁₋₆ alkyl. In some embodiments, R² is methyl. In some embodiments, R² is ethyl. In some embodiments, R² is propyl.

In some embodiments, —X¹═X²— is optionally substituted —CH₂—CH₂—. In some embodiments, —X¹═X²— is —CH₂—CH₂—.

### X³

In some embodiments, X³ is —N═ or —CR³═, wherein R³ is as described herein. In some embodiments, X³ is —N═. In some embodiments, X³ is —CR³═ wherein R³ is as described herein. In some embodiments, X³ is —CH═.

In some embodiments, R³ is R′, OR′, halogen, CN, —NO₂, or —N(R′)₂, wherein each variable is independently as described herein. In some embodiments, R³ is halogen. In some embodiments, R³ is F. In some embodiments, R³ is C₁. In some embodiments, R³ is Br. In some embodiments, R³ is I. In some embodiments, R³ is —CN. In some embodiments, R³ is —NO₂. In some embodiments, R³ is —OR′ wherein R′ is as described herein. In some embodiments, R³ is —N(R′)₂ wherein R′ is as described herein.

In some embodiments, R³ is R′ described herein. In some embodiments, R³ is R as described herein. In some embodiments, R³ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R³ is H. In some embodiments, R³ is optionally substituted C₁₋₆ aliphatic. In some embodiments, R³ is C₁₋₆ alkyl. In some embodiments, R³ is methyl. In some embodiments, R³ is ethyl.

In some embodiments, R³ is optionally substituted 6-10 membered aryl. In some embodiments, R³ is optionally substituted phenyl. In some embodiments, R³ is phenyl.

In some embodiments, R³ is optionally substituted 5-10 membered heteroaryl having 1-6 heteroatoms. In some embodiments, each heteroatom is independently selected from nitrogen, oxygen and sulfur.

In some embodiments, R³ is optionally substituted C₃-C₈ cycloalkyl. In some embodiments, R³ is optionally substituted

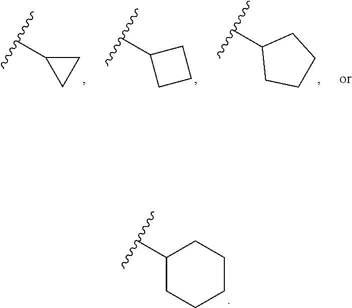

In some embodiments, R³ is optionally substituted

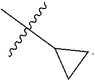 [SMILES: *C1CC1]

In some embodiments, R³ is optionally substituted

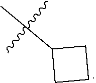 [SMILES: *C1CCC1]

In some embodiments, R³ is optionally substituted

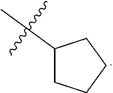 [SMILES: *C1CCCC1]

In some embodiments, R³ is optionally substituted

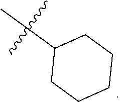 [SMILES: *C1CCCCC1]

In some embodiments, R³ is

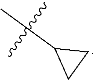 [SMILES: *C1CC1]

In some embodiments, R³ is

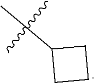 [SMILES: *C1CCC1]

In some embodiments, R³ is

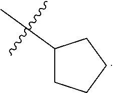 [SMILES: *C1CCCC1]

In some embodiments, R³ is

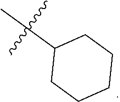 [SMILES: *C1CCCCC1]

### X⁴

In some embodiments, X⁴ is —N═ or —CR⁴═, wherein R⁴ is as described herein. In some embodiments, X⁴ is —N═. In some embodiments, X⁴ is —CR⁴═ wherein R⁴ is as described herein. In some embodiments, X⁴ is —CH═.

In some embodiments, R⁴ is R′, —OR′, halogen, —CN, —NO₂, or —N(R′)₂, wherein each variable is independently as described herein. In some embodiments, R⁴ is halogen. In some embodiments, R⁴ is F. In some embodiments, R⁴ is C₁. In some embodiments, R⁴ is Br. In some embodiments, R⁴ is I. In some embodiments, R⁴ is CN. In some embodiments, R⁴ is —OR′ wherein R′ is as described herein. In some embodiments, R⁴ is —NO₂. In some embodiments, R⁴ is —N(R′)₂ wherein R′ is as described herein.

In some embodiments, R⁴ is R′ described herein. In some embodiments, R⁴ is R as described herein. In some embodiments, R⁴ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁴ is H. In some embodiments, R⁴ is optionally substituted C₁₋₆ aliphatic. In some embodiments, R⁴ is C₁₋₆ alkyl. In some embodiments, R⁴ is methyl. In some embodiments, R⁴ is ethyl.

In some embodiments, R⁴ is optionally substituted 6-10 membered aryl. In some embodiments, R⁴ is optionally substituted phenyl. In some embodiments, R⁴ is phenyl.

In some embodiments, R⁴ is optionally substituted 5-10 membered heteroaryl having 1-6 heteroatoms. In some embodiments, each heteroatom is independently selected from nitrogen, oxygen and sulfur.

In some embodiments, R⁴ is optionally substituted C₃-C₈ cycloalkyl. In some embodiments, R⁴ is optionally substituted

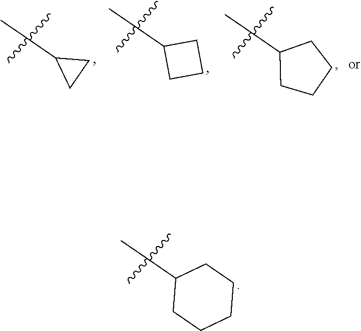

In some embodiments, R⁴ is optionally substituted

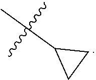 [SMILES: *C1CC1]

In some embodiments, R⁴ is optionally substituted

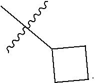 [SMILES: *C1CCC1]

In some embodiments, R⁴ is optionally substituted

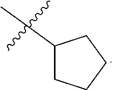 [SMILES: *C1CCCC1]

In some embodiments, R⁴ is optionally substituted

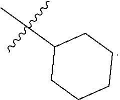 [SMILES: *C1CCCCC1]

In some embodiments, R⁴ is

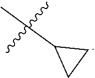 [SMILES: *C1CC1]

In some embodiments, R⁴ is

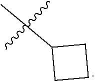 [SMILES: *C1CCC1]

In some embodiments, R⁴ is

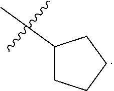 [SMILES: *C1CCCC1]

In some embodiments, R⁴ is

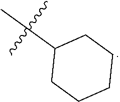 [SMILES: *C1CCCCC1]

In some embodiments, X³ is —N═ and X⁴ is —N═. In some embodiments, X³ is —C═ and X⁴ is —C═. In some embodiments, X³ is —C═ and X⁴ is —N═. In some embodiments, X³ is —N═ and X⁴ is —C═.

### R⁵

In some embodiments, R⁵ is R′, —OR′, halogen, —CN, —NO₂, or —N(R′)₂, wherein each variable is independently as described herein. In some embodiments, R⁵ is halogen. In some embodiments, R⁵ is F. In some embodiments, R⁵ is C₁. In some embodiments, R⁵ is Br. In some embodiments, R⁵ is I. In some embodiments, R⁵ is —CN. In some embodiments, R⁵ is —NO₂. In some embodiments, R⁵ is —OR′ wherein R′ is as described herein. In some embodiments, R⁵ is —N(R′)₂ wherein R′ is as described herein.

In some embodiments, R⁵ is R′ described herein. In some embodiments, R⁵ is R as described herein. In some embodiments, R⁵ is H or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic. In some embodiments, R⁵ is H. In some embodiments, R⁵ is optionally substituted C₁₋₆ aliphatic. In some embodiments, R⁵ is C₁₋₆ alkyl. In some embodiments, R⁵ is methyl. In some embodiments, R⁵ is ethyl.

In some embodiments, R⁵ is optionally substituted 6-10 membered aryl. In some embodiments, R⁵ is optionally substituted phenyl. In some embodiments, R⁵ is phenyl.

In some embodiments, R⁵ is optionally substituted 5-10 membered heteroaryl having 1-6 heteroatoms. In some embodiments, each heteroatom is independently selected from nitrogen, oxygen and sulfur.

In some embodiments, R⁵ is optionally substituted C₃-C₈ cycloalkyl. In some embodiments, R⁵ is optionally substituted

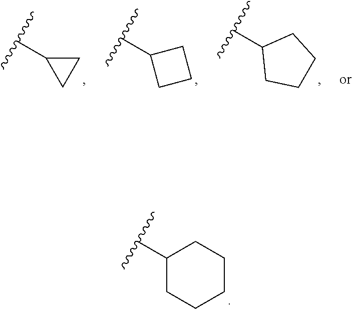

In some embodiments, R⁵ is optionally substituted

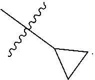 [SMILES: *C1CC1]

In some embodiments, R⁵ is optionally substituted

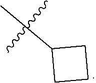 [SMILES: *C1CCC1]

In some embodiments, R⁵ is optionally substituted

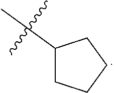 [SMILES: *C1CCCC1]

In some embodiments, R⁵ is optionally substituted

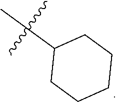 [SMILES: *C1CCCCC1]

In some embodiments, R⁵ is

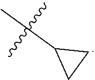 [SMILES: *C1CC1]

In some embodiments, R⁵ is

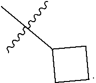 [SMILES: *C1CCC1]

In some embodiments, R⁵ is

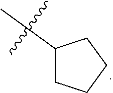 [SMILES: *C1CCCC1]

In some embodiments, R⁵ is

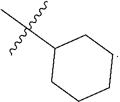 [SMILES: *C1CCCCC1]

### L

In some embodiments, L is optionally substituted —(CH₂)ₘ— wherein m is 1 or 2. In some embodiments, L is optionally substituted —CH₂—. In some embodiments, L is —CH(CH₃)—. In some embodiments, L is optionally substituted —(CH₂)₂—. In some embodiments, L is —CH(CH₃)—. In some embodiments, L is —CH₂—. In some embodiments, L is —(CH₂)₂—. In some embodiments, each substitutable hydrogen is independently and optionally substituted by R′, —OR′, halogen, —CN, —NO₂, or —N(R′)₂, wherein R′ is —H, C₁₋₄ alkyl or C₁₋₄ haloalkyl.

### Ring A

In some embodiments, Ring A is an optionally substituted ring selected from a 6-10 membered aryl ring and a 5-10 membered heteroaryl ring having 1-6 heteroatoms. In some embodiments, Ring A is optionally substituted 6-10 membered aryl ring. In some embodiments, Ring A is optionally substituted phenyl. In some embodiments, Ring A is phenyl. In some embodiments, Ring A is optionally substituted

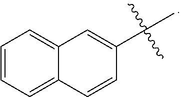 [SMILES: *c1ccc2ccccc2c1]

In some embodiments, Ring A is optionally substituted

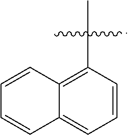 [SMILES: *c1cccc2ccccc12]

In some embodiments, Ring A is

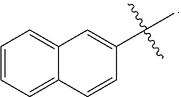 [SMILES: *c1ccc2ccccc2c1]

In some embodiments, Ring A is

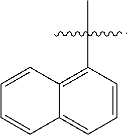 [SMILES: *c1cccc2ccccc12]

In some embodiments, Ring A is

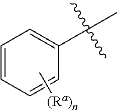

wherein each R^{a} is independently —R′, —OR′, halogen, —CN, —NO₂, or —N(R′)₂, each R′ is independently as described herein and n is 1, 2, 3, 4, or 5.

In some embodiments, Ring A is

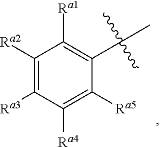

wherein each of R^{a1}, R^{a2}, R^{a3}, R^{a4}, and R^{a5} is independently R^{a} as described herein. In some embodiments, R^{a} is R^{a1} as described herein. In some embodiments, R^{a} is R^{a2} as described herein. In some embodiments, R^{a} is R^{a3} as described herein. In some embodiments, R^{a} is R^{a4} as described herein. In some embodiments, R^{a} is R^{a5} as described herein.

In some embodiments, R^{a1} is H. In some embodiments, R^{a1} is halogen. In some embodiments, R^{a1} is F. In some embodiments, R^{a1} is Cl. In some embodiments, R^{a1} is Br. In some embodiments, R^{a1} is I. In some embodiments, R^{a1} is —CN. In some embodiments, R^{a1} is —NO₂. In some embodiments, R^{a1} is —OR′ wherein R′ is as described herein. In some embodiments, R^{a1} is —OH. In some embodiments, R^{a1} is —OMe. In some embodiments, R^{a1} is —OCF₃. In some embodiments, R^{a1} is —N(R′)₂ wherein each R′ is independently as described herein.

In some embodiments, R^{a1} is R′. In some embodiments, R^{a1} is R. In some embodiments, R^{a1} is optionally substituted group selected from C₁-C₆ aliphatic. In some embodiments, R^{a1} is C₁-C₆ aliphatic optionally substituted by halogen. In some embodiments, R^{a1} is C₁-C₆ alkyl group optionally substituted by halogen. In some embodiments, Ral is methyl group optionally substituted by halogen. In some embodiments, R^{a1} is —CF₃.

In some embodiments, R^{a2} is H. In some embodiments, R^{a2} is halogen. In some embodiments, R^{a2} is F. In some embodiments, R^{a2} is C₁. In some embodiments, R^{a2} is Br. In some embodiments, R^{a2} is I. In some embodiments, R^{a2} is —CN. In some embodiments, R^{a2} is —NO₂. In some embodiments, R^{a2} is —OR′ wherein R′ is as described herein. In some embodiments, R^{a2} is —OH. In some embodiments, R^{a2} is —OMe. In some embodiments, R^{a2} is —OCF₃. In some embodiments, R^{a2} is —N(R′)₂ wherein each R′ is independently as described herein.

In some embodiments, R^{a2} is R′. In some embodiments, R^{a2} is R. In some embodiments, R^{a2} is optionally substituted group selected from C₁-C₆ aliphatic. In some embodiments, R^{a2} is C₁-C₆ aliphatic optionally substituted by halogen. In some embodiments, R^{a2} is C₁-C₆ alkyl group optionally substituted by halogen. In some embodiments, R^{a2} is methyl group optionally substituted by halogen. In some embodiments, R^{a2} is —CF₃.

In some embodiments, R^{a3} is H. In some embodiments, R^{a3} is halogen. In some embodiments, R^{a3} is F. In some embodiments, R^{a3} is C₁. In some embodiments, R^{a3} is Br. In some embodiments, R^{a3} is I. In some embodiments, R^{a3} is —CN. In some embodiments, R^{a3} is —NO₂. In some embodiments, R^{a3} is —OR′ wherein R′ is as described herein. In some embodiments, R^{a3} is —OH. In some embodiments, R^{a3} is —OMe. In some embodiments, R^{a3} is —OCF₃. In some embodiments, R^{a3} is —N(R′)₂ wherein each R′ is independently as described herein.

In some embodiments, R^{a3} is R′. In some embodiments, R^{a3} is R. In some embodiments, R^{a3} is optionally substituted group selected from C₁-C₆ aliphatic. In some embodiments, R^{a3} is C₁-C₆ aliphatic optionally substituted by halogen. In some embodiments, R^{a3} is C₁-C₆ alkyl group optionally substituted by halogen. In some embodiments, R^{a3} is methyl group optionally substituted by halogen. In some embodiments, R^{a3} is —CF₃.

In some embodiments, R^{a4} is H. In some embodiments, R^{a4} is halogen. In some embodiments, R^{a4} is F. In some embodiments, R^{a4} is C₁. In some embodiments, R^{a4} is Br. In some embodiments, R^{a4} is I. In some embodiments, R^{a4} is —CN. In some embodiments, R^{a4} is —NO₂. In some embodiments, R^{a4} is —OR′ wherein R′ is as described herein. In some embodiments, R^{a4} is —OH. In some embodiments, R^{a4} is —OMe. In some embodiments, R^{a4} is —OCF₃. In some embodiments, R^{a4} is —N(R′)₂ wherein each R′ is independently as described herein.

In some embodiments, R^{a4} is R′. In some embodiments, R^{a4} is R. In some embodiments, R^{a4} is optionally substituted group selected from C₁-C₆ aliphatic. In some embodiments, R^{a4} is C₁-C₆ aliphatic optionally substituted by halogen. In some embodiments, R^{a4} is C₁-C₆ alkyl group optionally substituted by halogen. In some embodiments, R^{a4} is methyl group optionally substituted by halogen. In some embodiments, R^{a4} is —CF₃.

In some embodiments, R^{a5} is H. In some embodiments, R^{a5} is halogen. In some embodiments, R^{a5} is F. In some embodiments, R^{a5} is C₁. In some embodiments, R^{a5} is Br. In some embodiments, R^{a5} is I. In some embodiments, R^{a5} is —CN. In some embodiments, R^{a5} is —NO₂. In some embodiments, R^{a5} is —OR′ wherein R′ is as described herein. In some embodiments, R^{a5} is —OH. In some embodiments, R^{a5} is —OMe. In some embodiments, R^{a5} is —OCF₃. In some embodiments, R^{a5} is —N(R′)₂ wherein each R′ is independently as described herein.

In some embodiments, R^{a5} is R′. In some embodiments, R^{a5} is R. In some embodiments, R^{a5} is optionally substituted group selected from C₁-C₆ aliphatic. In some embodiments, R^{a5} is C₁-C₆ aliphatic optionally substituted by halogen. In some embodiments, R^{a5} is C₁-C₆ alkyl group optionally substituted by halogen. In some embodiments, R^{a5} is methyl group optionally substituted by halogen. In some embodiments, R^{a5} is —CF₃.

In some embodiments, Ring A is

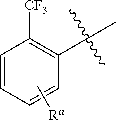

wherein R^{a} is —R′, —OR′, halogen, —CN, —NO₂, or —N(R′)₂ and each R′ is independently as described herein.

In some embodiments, Ring A is Ring A is

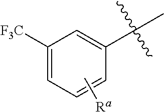

wherein R^{a} is —R′, —OR′, halogen, —CN, —NO₂, or —N(R′)₂ and each R′ is independently as described herein.

In some embodiments, Ring A is

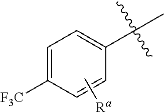

wherein R^{a} is —R′, —OR′, halogen, —CN, —NO₂, or —N(R′)₂ and each R′ is independently as described herein.

In some embodiments, Ring A is

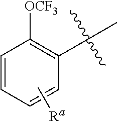

wherein R^{a} is —R′, —OR′, halogen, —CN, —NO₂, or —N(R′)₂ and each R′ is independently as described herein.

In some embodiments, Ring A is

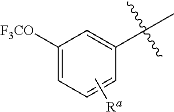

wherein R^{a} is —R′, —OR′, halogen, —CN, —NO₂, or —N(R′)₂ and each R′ is independently as described herein.

In some embodiments, Ring A is

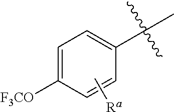

wherein R^{a} is —R′, —OR′, halogen, —CN, —NO₂, or —N(R′)₂ and each R′ is independently as described herein.

In some embodiments, Ring A is

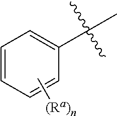

and R^{a} is —R′, —OR′, halogen, CN, —NO₂, or —N(R′)₂ and each R′ is independently as described herein and n is 1 or 2. In some embodiments, Ring A is

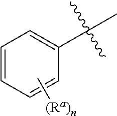

and R^{a} is halogen and n is 1 or 2.

In some embodiments, Ring A is

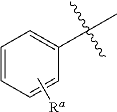

and R^{a} is —R′, —OR′, halogen, —CN, —NO₂, or —N(R′)₂ and each R′ is independently as described herein. In some embodiments, Ring A is

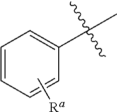

and R^{a} is halogen. In some embodiments, Ring A is

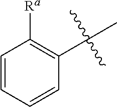

and R^{a} is halogen. In some embodiments, Ring A is

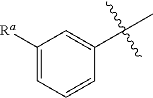

and R^{a} is halogen. In some embodiments, Ring A is

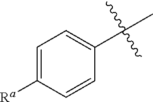

and R^{a} is halogen.

In some embodiments, Ring A is

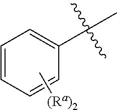

and each R^{a} is independently —R′, —OR′, halogen, —CN, —NO₂, or —N(R′)₂ and each R′ is independently as described herein. In some embodiments, Ring A is

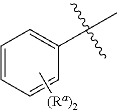

and each R^{a} is independently halogen. In some embodiments, Ring A is

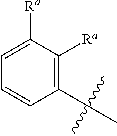

and each R^{a} is independently halogen.

In some embodiments, R^{a} is H. In some embodiments, R^{a} is halogen. In some embodiments, R^{a} is F. In some embodiments, R^{a} is Cl. In some embodiments, R^{a} is Br. In some embodiments, R^{a} is I. In some embodiments, R^{a} is —CN. In some embodiments, R^{a} is —NO₂. In some embodiments, R^{a} is —OR′ wherein R′ is as described herein. In some embodiments, R^{a} is —OH. In some embodiments, R^{a} is —OMe. In some embodiments, R^{a} is —OCF₃. In some embodiments, R^{a} is —N(R′)₂ wherein each R′ is independently as described herein.

In some embodiments, R^{a} is R′. In some embodiments, R^{a} is R. In some embodiments, R^{a} is optionally substituted group selected from C₁-C₆ aliphatic. In some embodiments, R^{a} is C₁-C₆ aliphatic optionally substituted by halogen. In some embodiments, R^{a} is C₁-C₆ alkyl group optionally substituted by halogen. In some embodiments, R^{a} is methyl group optionally substituted by halogen. In some embodiments, R^{a} is —CF₃.

In some embodiments, Ring A is selected from:

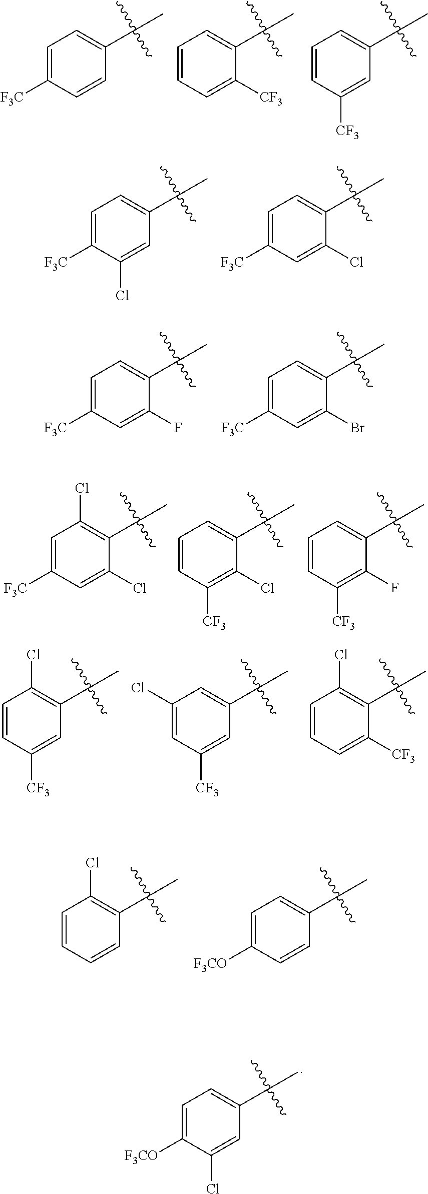

In some embodiments, Ring A is an optionally substituted 5-10 membered heteroaryl ring having 1-6 heteroatoms. In some embodiments, Ring A is an optionally substituted 5-6 membered heteroaryl ring having 1-4 heteroatoms. In some embodiments, Ring A is a 6 membered heteroaryl ring having 1-2 heteroatoms. In some embodiments, Ring A is a 6 membered heteroaryl ring having 1-2 heteroatoms independently selected from O, N, and S. In some embodiments, Ring A is a 6 membered heteroaryl ring having a heteroatom selected from O, N, and S. In some embodiments, Ring A is a 6 membered heteroaryl ring having a nitrogen atom. In some embodiments, Ring A is a 6 membered heteroaryl ring having 2 heteroatoms independently selected from O, N, and S. In some embodiments, Ring A is a 6 membered heteroaryl ring having 2 heteroatoms each of which is N. In some embodiments, Ring A is a 6 membered heteroaryl ring having 3 heteroatoms. In some embodiments, Ring A is a 6 membered heteroaryl ring having 3 heteroatoms independently selected from O, N, and S. In some embodiments, Ring A is a 6 membered heteroaryl ring having 3 heteroatoms wherein one is N. In some embodiments, Ring A is a 5 membered heteroaryl ring having 1-2 heteroatoms. In some embodiments, Ring A is a 5 membered heteroaryl ring having 1-2 heteroatoms independently selected from O, N, and S. In some embodiments, Ring A is a 5 membered heteroaryl ring having an oxygen atom. In some embodiments, Ring A is a 5 membered heteroaryl ring having a nitrogen atom. In some embodiments, Ring A is a 5 membered heteroaryl ring having a sulfur atom. In some embodiments, Ring A is a 5 membered heteroaryl ring having 2 heteroatoms independently selected from O, N, and S. In some embodiments, Ring A is a 5 membered heteroaryl ring having 2 heteroatoms one of which is N. In some embodiments, Ring A is a 5 membered heteroaryl ring having 2 heteroatoms independently selected from N and S. In some embodiments, Ring A′ is a 5 membered heteroaryl ring having 2 heteroatoms independently selected from N and O. In some embodiments, Ring A is a 5 membered heteroaryl ring having 2 heteroatoms independently selected from O and S. In some embodiments, Ring A is a 5 membered heteroaryl ring having 3 heteroatoms. In some embodiments, Ring A is a 5 membered heteroaryl ring having 3 heteroatoms independently selected from O, N, and S. In some embodiments, a ring has a single heteroatom. In some embodiments, a ring has two or more heteroatoms at least one of which is nitrogen. In some embodiments, each is nitrogen. In some embodiments, all heteroatoms are the same. In some embodiments, at least one heteroatom is different from the other heteroatom(s).

In some embodiments, Ring A is an optionally substituted 9 membered heteroaryl ring having 1-4 heteroatoms. In some embodiments, Ring A is an optionally substituted 9-membered heteroaryl ring having 4 heteroatoms independently selected from O, N, and S. In some embodiments, Ring A is an optionally substituted 9-membered heteroaryl ring having 3 heteroatoms independently selected from O, N, and S. In some embodiments, Ring A is an optionally substituted 9-membered heteroaryl ring having 3 heteroatoms one of which is N. In some embodiments, Ring A is an optionally substituted 9-membered heteroaryl ring having 3 heteroatoms independently selected from O and N. In some embodiments, Ring A is an optionally substituted 9-membered heteroaryl ring having 3 heteroatoms independently selected from O and S. In some embodiments, Ring A is an optionally substituted 9-membered heteroaryl ring having 3 heteroatoms independently selected from N and S. In some embodiments, Ring A is an optionally substituted 9-membered heteroaryl ring having 2 heteroatoms independently selected from O, N, and S. In some embodiments, Ring A is an optionally substituted 9-membered heteroaryl ring having 2 heteroatoms one of which is N. In some embodiments, Ring A is an optionally substituted 9-membered heteroaryl ring having 2 heteroatoms independently selected from O and N. In some embodiments, Ring A is an optionally substituted 9-membered heteroaryl ring having 2 heteroatoms independently selected from O and S. In some embodiments, Ring A is an optionally substituted 9-membered heteroaryl ring having 2 heteroatoms independently selected from N and S. In some embodiments, Ring A is an optionally substituted 9-membered heteroaryl ring having 2 heteroatoms both of which are N. In some embodiments, Ring A is an optionally substituted 9-membered heteroaryl ring having 1 heteroatom selected from O, N, and S. In some embodiments, Ring A is an optionally substituted 9-membered heteroaryl ring having a nitrogen atom. In some embodiments, Ring A is an optionally substituted 9-membered heteroaryl ring having an oxygen atom. In some embodiments, Ring A is an optionally substituted 9-membered heteroaryl ring having an sulfur atom. In some embodiments, a ring has a single heteroatom. In some embodiments, a ring has two or more heteroatoms at least one of which is nitrogen. In some embodiments, each is nitrogen. In some embodiments, all heteroatoms are the same. In some embodiments, at least one heteroatom is different from the other heteroatom(s).

In some embodiments, Ring A is an optionally substituted 10 membered heteroaryl ring having 1-4 heteroatoms. In some embodiments, Ring A is an optionally substituted 10-membered heteroaryl ring having 4 heteroatoms independently selected from O, N, and S. In some embodiments, Ring A is an optionally substituted 10-membered heteroaryl ring having 3 heteroatoms independently selected from O, N, and S. In some embodiments, Ring A is an optionally substituted 10-membered heteroaryl ring having 3 heteroatoms all of which are N. In some embodiments, Ring A is an optionally substituted 10-membered heteroaryl ring having 2 heteroatoms independently selected from O, N, and S. In some embodiments, Ring A is an optionally substituted 10-membered heteroaryl ring having 2 heteroatoms one of which is N. In some embodiments, Ring A is an optionally substituted 10-membered heteroaryl ring having 2 heteroatoms independently selected from O and N. In some embodiments, Ring A is an optionally substituted 10-membered heteroaryl ring having 2 heteroatoms independently selected from O and S. In some embodiments, Ring A is an optionally substituted 10-membered heteroaryl ring having 2 heteroatoms independently selected from N and S. In some embodiments, Ring A is an optionally substituted 10-membered heteroaryl ring having 2 heteroatoms both of which are N. In some embodiments, Ring A is an optionally substituted 10-membered heteroaryl ring having 1 heteroatom selected from O, N, and S. In some embodiments, Ring A is an optionally substituted 10-membered heteroaryl ring having a nitrogen atom. In some embodiments, Ring A is an optionally substituted 10-membered heteroaryl ring having an oxygen atom. In some embodiments, Ring A is an optionally substituted 10-membered heteroaryl ring having a sulfur atom. In some embodiments, a ring has a single heteroatom. In some embodiments, a ring has two or more heteroatoms at least one of which is nitrogen. In some embodiments, each is nitrogen. In some embodiments, all heteroatoms are the same. In some embodiments, at least one heteroatom is different from the other heteroatom(s).

### R′

Various variables can be R′ as described herein. In some embodiments, R′ is hydrogen.

In some embodiments, R′ is R as described herein. In some embodiments, R′ is —OR wherein R is as described herein. In some embodiments, R′ is —C(O)R wherein R is as described herein. In some embodiments, R′ is —C(O)OR wherein R is as described herein. In some embodiments, R′ is —S(O)₂R wherein R is as described herein.

### R

Various variables can be R as described herein. Various embodiments for R are extensively described herein, including in various sections for other variables that can be R (e.g., R¹, R², R′, etc.).

In some embodiments, R is —H. In some embodiments, R is not —H.

In some embodiments, each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic.

In some embodiments, R is optionally substituted C₁₋₆ aliphatic. In some embodiments, R is optionally substituted C₁₋₆ alkyl. In some embodiments, R is optionally substituted methyl. In some embodiments, R is optionally substituted ethyl. In some embodiments, R is optionally substituted n-propyl. In some embodiments, R is optionally substituted isopropyl. In some embodiments, R is n-butyl. In some embodiments, R is t-butyl. In some embodiments, R is pentyl. In some embodiments, R is hexyl.

In some embodiments, R is optionally substituted C₁₋₆ heteroaliphatic having 1-3 (e.g., 1, 2, or 3) heteroatoms independently selected from oxygen, nitrogen, sulfur, phosphorus and silicon. In some embodiments, R is optionally substituted C₁₋₆ heteroaliphatic having 1-3 (e.g., 1, 2, or 3) heteroatoms independently selected from nitrogen, oxygen and sulfur. In some embodiments, a heteroatom is nitrogen. In some embodiments, a heteroatom is oxygen. In some embodiments, a heteroatom is sulfur.

In some embodiments, R is optionally substituted C₃₋₁₀ (e.g., C₄₋₁₀, C₃₋₉, C₃₋₇, or 3, 4, 5, 6, 7, 8, 9, or 10-membered) cycloaliphatic. In some embodiments, a cycloaliphatic group is a cycloalkyl group. In some embodiments, a cycloaliphatic group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered cycloaliphatic ring. In some embodiments, a cycloaliphatic group is saturated. In some embodiments, it is partially unsaturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted cycloheptyl. In some embodiments, R is cyclopropyl. In some embodiments, R is cyclobutyl. In some embodiments, R is cyclopentyl. In some embodiments, R is cyclohexyl. In some embodiments, R is cycloheptyl.

In some embodiments, R is optionally substituted 3-10 (e.g., 3-9, 3-6, 3-5, or 3, 4, 5, 6, 7, 8, 9, or 10, etc.) membered heterocyclyl having 1-4 (e.g., 1, 2, 3, or 4, etc.) heteroatoms independently selected from oxygen, nitrogen, sulfur, phosphorus and silicon. In some embodiments, R is optionally substituted 3-10 (e.g., 3-9, 3-6, 3-5, or 3, 4, 5, 6, 7, 8, 9, or 10, etc.) membered heterocyclyl having 1-4 (e.g., 1, 2, 3, or 4, etc.) heteroatoms independently selected from oxygen, nitrogen and sulfur. In some embodiments, a heterocyclyl group is monocyclic. In some embodiments, it is bicyclic. In some embodiments, it is polycyclic. In some embodiments, each monocyclic unit is independently a 3-10 (e.g., 3, 4, 5, 6, 7, 8, 9, or 10, etc.) membered heterocyclyl ring having 1-4 (e.g., 1, 2, 3, or 4 etc.) heteroatoms independently selected from nitrogen, oxygen and sulfur. In some embodiments, a heterocyclyl group is saturated. In some embodiments, it is partially unsaturated. In some embodiments, a heterocyclyl ring has one heteroatom. In some embodiments, a heterocyclyl ring has two or more heteroatoms. In some embodiments, a heterocyclyl ring has three or more heteroatoms. In some embodiments, a heterocyclyl ring has four or more heteroatoms. In some embodiments, a heteroatom is nitrogen. In some embodiments, a heteroatom is oxygen. In some embodiments, a heteroatom is sulfur.

In some embodiments, R is optionally substituted C₆₋₁₀ (e.g., C₆, C₁₀, etc.) aryl. In some embodiments, an aryl ring is monocyclic. In some embodiments, an aryl ring is bicyclic. In some embodiments, an aryl ring is polycyclic. In some embodiments, each monocyclic unit is independently a 6-membered aromatic ring. In some embodiments, R is optionally substituted phenyl. In some embodiments, R is phenyl. In some embodiments, R is optionally substituted 10-membered aryl. In some embodiments, R is optionally substituted naphthyl. In some embodiments, R is naphthyl.

In some embodiments, R is optionally substituted 5-10 (e.g., 5-9, or 5, 6, 7, 8, 9, or 10 etc.) membered heteroaryl having 1-6 (e.g., 1-6, 1-5, 1-4, or 1, 2, 3, 4, 5, or 6 etc.) heteroatoms independently selected from oxygen, nitrogen, sulfur, phosphorus and silicon. In some embodiments, R is 5-10 (e.g., 5-9, or 5, 6, 9, 10 etc.) membered heteroaryl having 1-4 (e.g., 1, 2, 3, or 4, etc.) heteroatoms independently selected from nitrogen, oxygen and sulfur. In some embodiments, a heteroaryl ring is monocyclic. In some embodiments, a heteroaryl ring is bicyclic. In some embodiments, a heteroaryl ring is polycyclic. In some embodiments, each monocyclic unit is independently a 5- or 6-membered aromatic ring having 0-4 heteroatoms, e.g., independently selected from nitrogen, oxygen and sulfur, wherein at least one monocyclic unit contains 1~4 heteroatoms. In some embodiments, R is optionally substituted 5-membered monocyclic heteroaryl having 1-4 heteroatoms independently selected from nitrogen, oxygen and sulfur. In some embodiments, R is optionally substituted 6-membered monocyclic heteroaryl having 1-4 heteroatoms independently selected from nitrogen, oxygen and sulfur. In some embodiments, R is optionally substituted 8-membered bicyclic heteroaryl having 1-4 heteroatoms independently selected from nitrogen, oxygen and sulfur. In some embodiments, R is optionally substituted 9-membered bicyclic heteroaryl having 1-4 heteroatoms independently selected from nitrogen, oxygen and sulfur. In some embodiments, R is optionally substituted 10-membered bicyclic heteroaryl having 1-4 heteroatoms independently selected from nitrogen, oxygen and sulfur. In some embodiments, a heteroaryl ring has one heteroatom. In some embodiments, a heteroaryl ring has two or more heteroatoms. In some embodiments, a heteroaryl ring has three or more heteroatoms. In some embodiments, a heteroaryl ring has four or more heteroatoms. In some embodiments, a heteroatom is nitrogen. In some embodiments, a heteroatom is oxygen. In some embodiments, a heteroatom is sulfur.

In some embodiments, R is optionally substituted C₆₋₁₀ (e.g., C₆, C₁₀, etc.) aryl-C₁₋₆ aliphatic, wherein the aryl and aliphatic are independently as described herein. In some embodiments, R is optionally substituted C₆₋₁₀ aryl-C₁₋₆ alkyl.

In some embodiments, R is optionally substituted 5-10 (e.g., 5-9, or 5, 6, 7, 8, 9, or 10 etc.) membered heteroaryl having 1-6 (e.g., 1, 2, 3, 4, 5, or 6) heteroatoms-C₁₋₆ aliphatic wherein the heteroaryl and aliphatic are independently as described herein. In some embodiments, R is optionally substituted 5-10 membered heteroaryl having 1-5 heteroatoms-C₁₋₆ aliphatic. In some embodiments, R is optionally substituted 5-6 membered heteroaryl having 1-4 heteroatoms-C₁₋₆ aliphatic. In some embodiments, R is optionally substituted 5-10 membered heteroaryl having 1-6 (e.g., 1, 2, 3, 4, 5, or 6) heteroatoms-C₁₋₆ alkyl wherein the heteroaryl and alkyl are independently as described herein. In some embodiments, R is optionally substituted 5-10 membered heteroaryl having 1-5 heteroatoms-C₁₋₆ alkyl. In some embodiments, R is optionally substituted 5-6 membered heteroaryl having 1-4 heteroatoms-C₁₋₆ alkyl. Various suitable heteroaryl and aliphatic groups are as described herein.

In some embodiments, two R groups on the same atom are optionally and independently taken together with the atom to form an optionally substituted 3-10 (e.g., 3, 4, 5, 6, 7, 8, 9, or 10, etc.) membered ring having, in addition to the atom, 0-4 (e.g., 0, 1, 2, 3, or 4) heteroatoms. In some embodiments, two R groups on two atoms are optionally and independently taken together with their intervening atoms to form an optionally substituted 3-10 (e.g., 3, 4, 5, 6, 7, 8, 9, or 10, etc.) membered ring having, in addition to the intervening atoms, 0-4 (e.g., 0, 1, 2, 3, or 4) heteroatoms

As described herein, in various instances, two R groups, or two groups that are or can be R (e.g., R′, R², R³, etc.,), can be taken together with their intervening atom(s) to form an optionally substituted ring as described herein. In some embodiments, a formed ring is substituted (in addition to groups attached to the intervening atom(s)). In some embodiments, a formed ring is unsubstituted. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is saturated. In some embodiments, a formed ring is partially unsaturated. In some embodiments, a formed ring is aromatic. In some embodiments, a formed ring is monocyclic. In some embodiments, it is bicyclic. In some embodiments, it is polycyclic. In some embodiments, each monocyclic unit is independently a 3-10 (e.g., 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4) heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, a formed ring has, in addition to the intervening atom(s), 0-4 (e.g., 0, 1, 2, 3, or 4, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In certain embodiments, the present disclosure provides a compound of Formula (II), or a salt thereof, wherein each of X¹, X², X³, X⁴, R⁵, L, and Ring A is independently as described herein.

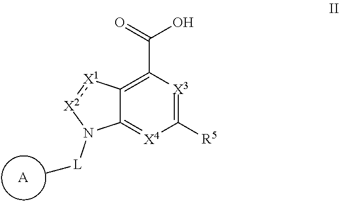

In certain embodiments, the present disclosure provides a compound of Formula (III), or a salt thereof, wherein each of X¹, X², X³, X⁴, R⁵, L, and Ring A is independently as described herein.

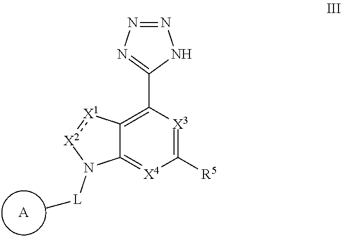

In certain embodiments, the present disclosure provides a compound of Formula (IV), or a salt thereof, wherein each of R³, R⁴, R⁵, L, and Ring A is independently as described herein.

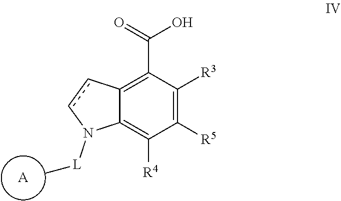

In certain embodiments, the present disclosure provides a compound of Formula (V), or a salt thereof, wherein each of R³, R⁴, R⁵, L, and Ring A is independently as described herein.

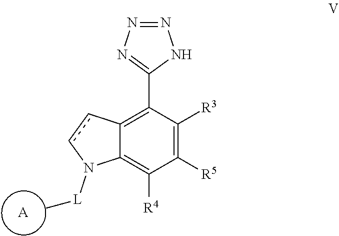

In some embodiments, a provided compound is a compound selected from Compounds 1-55 or a salt thereof in the Exemplification.

In some embodiments, each heteroatom is independently selected from oxygen, nitrogen, sulfur, phosphorus and silicon. In some embodiments, each heteroatom is independently selected from oxygen, nitrogen and sulfur.

In some embodiments, one or more isotopes may be utilized or enriched in compounds of the present disclosure at one or more locations. For example, in some embodiments, deuterium is utilized or enriched at one or more positions. In some embodiments, an enrichment is about or at least about 5%, 10%, 15%, 20%, 25%, 30%, 35%, 40%, 45%, 50%, 60%, 70%, 80%, 85%, 90%, 95%, or 99% more than a natural abundance as applicable. In some embodiments, a level of an isotope at a position is about or at least about 5%, 10%, 15%, 20%, 25%, 30%, 35%, 40%, 45%, 50%, 60%, 70%, 80%, 85%, 90%, 95%, or 99% of all compound molecules.

Among other things, the present disclosure provides stereochemically pure, e.g., enantiomerically pure, compounds with purities as described herein. In some embodiments, a compound is stereochemically pure. In some embodiments, a compound is enantiomerically pure. In some embodiments, a provided composition is enriched for one enantiomer over the other, or with respect to a chiral center, for one configuration over the other. For example, in some embodiments, the percentage of an enantiomer, or one configuration with respect to a chiral center, is about or at least about 60%, 65%, 70%, 75%, 80%, 85%, 90%, 95%, 96%, 97%, 98%, 99%, or 99.5%. In some embodiments, it is about or at least about 60%. In some embodiments, it is about or at least about 65%. In some embodiments, it is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about or at least about 96%. In some embodiments, it is about or at least about 97%. In some embodiments, it is about or at least about 98%. In some embodiments, it is about or at least about 99%. In some embodiments, it is about or at least about 99.5%. In some embodiments, the (S) enantiomer or configuration is enriched. In some embodiments, the (R) enantiomer or configuration is enriched.

### Methods of Preparing

In some embodiments, the present disclosure provides various technologies, e.g., reagents, intermediates, conditions, etc. for preparing compounds and compositions as described herein. Those skilled in the art appreciate that many technologies are available and can be utilized in accordance with the present disclosure.

As appreciated by those skilled in the art, in chemical reactions various groups, e.g., hydroxyl, amino, carboxyl, etc. may be protected to avoid undesired reactions. Many technologies for protection/deprotection are available to those skilled in the art and may be utilized in accordance with the present disclosure. Certain such technologies are described herein including exemplified in the Examples.

Various chemical reactions are typically performed in a solvent. In some embodiments, a reaction is performed in a single solvent, e.g., DCM, THF, Et₂O, EtOH, toluene, etc. In some embodiments, a reaction is performed in a mixture of two or more solvents. In some embodiments, a solvent is polar. In some embodiments, a solvent is non-polar. In some embodiments, a solvent is protic. In some embodiments, a solvent is non-protic. In some embodiments, a solvent is polar but is not protic. Suitable solvents for various reactions are available to those skilled in the art and can be utilized in accordance with the present disclosure.

In some embodiments, a reaction is conducted under an inert atmosphere, e.g., N₂, Ar, etc. In some embodiments, a reaction is conducted with exposure to air. In some embodiments, a reaction is conducted under anhydrous conditions, e.g., with reagents, solvents, vessels, etc., properly dried. In some embodiments, a reaction is conducted in the presence of significant of water (e.g., about or more than about 0.1, 0.5, or 1 equivalent).

In some embodiments, reactions are performed, or are performed for periods of time, at temperatures that are higher or lower than or about a standard ambient temperature (25° C.). In some embodiments, a reaction temperature is lower than a standard ambient temperature. In some embodiments, a temperature is about or no more than about −78, −60, −50, −40, −30, −20, −10, 0 or 10° C. In some embodiments, a temperature is about or no more than about 10° C. In some embodiments, a temperature is about or no more than about 15° C. In some embodiments, a temperature is about or no more than about 20° C. In some embodiments, a reaction temperature is about a standard ambient temperature. In some embodiments, a reaction temperature is higher than a standard ambient temperature. In some embodiments, a reaction temperature is about or at least about 35, 40, 50, 60, 70, 80, 90, 100, or 100° C. In some embodiments, a reaction comprises refluxing in a boiling solvent system, e.g., in ether, toluene, etc. In some embodiments, temperature changes during a reaction process, e.g., increasing from a lower temperature to a higher temperature, decreasing from a higher temperature to a lower temperature, or both.

In some embodiments, a product is selectively produced over another potential product. In some embodiments, a product is produced with chemoselectivity, stereoselectivity and/or regioselectivity. In some embodiments, a selectivity is presented as a ratio, e.g., of one product over another. In some embodiments, a ratio is about or at least about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 40:1, 50:1, 60:1, 70:1, 80:1, 90:1, 100:1, 200:1, 500:1 or more.

Reactions may be performed for a variety of time lengths. In some embodiments, reactions complete instantly. In some embodiments, reaction times varies from minutes to hours to days, e.g., 5, 10, 15, 20, 30, 45 minutes, or 1, 1.5, 2, 3, 4, 5, 6, 7, 8, 9, 10, 12, 15, 18, 20 or 22 hours, or one or two days or longer. Those skilled in the art can use various technologies to determine when to terminate reactions, e.g., based on consumption of starting materials, products formation, by-products formation, etc.

In some embodiments, the present disclosure provides compounds of high purity. In some embodiments, purity of a compound is or greater than about 85%, 90%, 91%, 92%, 93%, 94%, 95%, 96%, 97%, 98%, 99%, 99.7%, or 99.9%. In some embodiments, purity of a compound is or greater than about 80%. In some embodiments, purity of a compound is or greater than about 85%. In some embodiments, purity of a compound is or greater than about 90%. In some embodiments, purity of a compound is or greater than about 95%. In some embodiments, purity of a compound is or greater than about 96%. In some embodiments, purity of a compound is or greater than about 97%. In some embodiments, purity of a compound is or greater than about 98%. In some embodiments, purity of a compound is or greater than about 99%. In some embodiments, purity of a compound is or greater than about 99.5%. In some embodiments, purity of a compound is or greater than about 99.7%. In some embodiments, purity of a compound is or greater than about 99.9%.

In some embodiments, the present disclosure provides compounds of high stereochemical purity. In some embodiments, stereochemical purity of a compound is or greater than about 85%, 90%, 91%, 92%, 93%, 94%, 95%, 96%, 97%, 98%, 99%, 99.7%, or 99.9%. In some embodiments, stereochemical purity of a compound is or greater than about 80%. In some embodiments, stereochemical purity of a compound is or greater than about 85%. In some embodiments, stereochemical purity of a compound is or greater than about 90%. In some embodiments, stereochemical purity of a compound is or greater than about 95%. In some embodiments, stereochemical purity of a compound is or greater than about 96%. In some embodiments, stereochemical purity of a compound is or greater than about 97%. In some embodiments, stereochemical purity of a compound is or greater than about 98%. In some embodiments, stereochemical purity of a compound is or greater than about 99%. In some embodiments, stereochemical purity of a compound is or greater than about 99.5%. In some embodiments, stereochemical purity of a compound is or greater than about 99.7%. In some embodiments, stereochemical purity of a compound is or greater than about 99.9%.

In some embodiments, the present disclosure provides compounds of high enantiomeric purity. In some embodiments, enantiomeric purity of a compound is or greater than about 85%, 90%, 91%, 92%, 93%, 94%, 95%, 96%, 97%, 98%, 99%, 99.7%, or 99.9%. In some embodiments, enantiomeric purity of a compound is or greater than about 80%. In some embodiments, enantiomeric purity of a compound is or greater than about 85%. In some embodiments, enantiomeric purity of a compound is or greater than about 90%. In some embodiments, enantiomeric purity of a compound is or greater than about 95%. In some embodiments, enantiomeric purity of a compound is or greater than about 96%. In some embodiments, enantiomeric purity of a compound is or greater than about 97%. In some embodiments, enantiomeric purity of a compound is or greater than about 98%. In some embodiments, enantiomeric purity of a compound is or greater than about 99%. In some embodiments, enantiomeric purity of a compound is or greater than about 99.5%. In some embodiments, enantiomeric purity of a compound is or greater than about 99.7%. In some embodiments, enantiomeric purity of a compound is or greater than about 99.9%.

Stereochemically pure, e.g., enantiomerically pure, compounds and compositions can be prepared utilizing various technologies in accordance with the present disclosure. For example, in some embodiments, they can be prepared through separation including chiral separation; in some embodiments, they can be prepared through stereoselective synthesis.

For example, in some embodiments, the present disclosure provides a method, comprising:
reacting a compound of formula B3

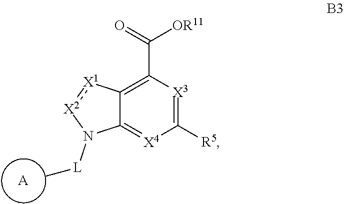

or a salt thereof to provide a compound having the structure of formula II:

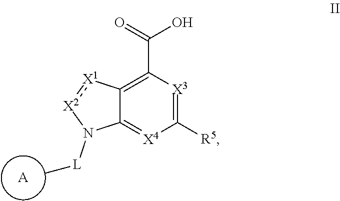

or a salt thereof, wherein each variable is independently as described herein.

In some embodiments, the present disclosure provides a method, comprising:
reacting a compound having the structure of formula B1:

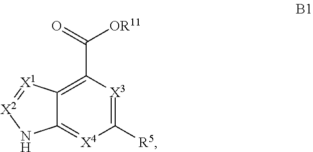

or a salt thereof with a compound having the structure of

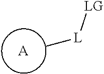

or a salt thereof, wherein LG is a leaving group to provide a compound having the structure of formula B3:

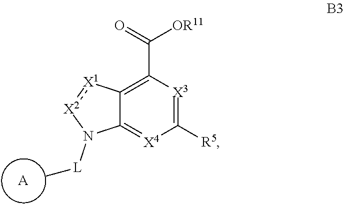

or a salt thereof, wherein each variable is independently as described herein.

In some embodiments, a method is described in Scheme 1 as an example:

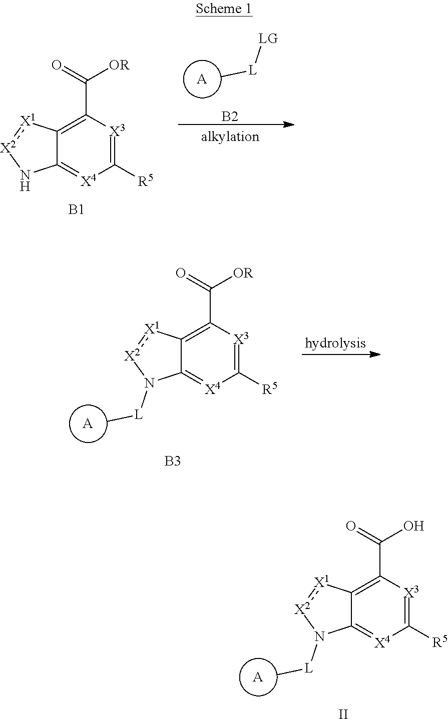

A method for preparing provided compounds, e.g., compounds of formula II or salts thereof, is illustrated in Scheme 1 as an example, wherein each variable is independently as described herein. In some embodiments, treatment of compound of formula B1 or a salt thereof, wherein X¹, X², X³, X⁴, R⁵ and R¹¹ are defined herein, and compound of formula B2 or a salt thereof, wherein Ring A and L are defined herein and LG is a leaving group, under alkylation conditions such as Cs₂CO₃/DMF provides alkylated compound of formula B3 or a salt thereof. Various leaving groups may be utilized in accordance with the present disclosure. For example, in some embodiments, a leaving group is a halogen. In some embodiments, LG is Cl. In some embodiments, LG is Br. In some embodiments, LG is I. In some embodiments, LG is —OS(O)₂R wherein R is as described herein and is not H. In some embodiments, R is optionally substituted C₁₋₆ aliphatic. In some embodiments, R is optionally substituted phenyl. Many suitable conditions may be utilized in accordance with the present disclosure. For example, in some embodiments, a condition is an alkylation condition. In some embodiments, a reaction is performed in the presence of a base. In some embodiments, a base is Cs₂CO₃. Hydrolysis of ester compound of formula B3 or a salt thereof under basic conditions such as LiOH, NaOH, or KOH affords compound of formula II or a salt thereof.

In some embodiments, the present disclosure provides a method, comprising:
reacting a compound of formula C3

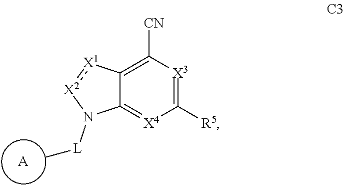

or a salt thereof to provide a compound having the structure of formula III:

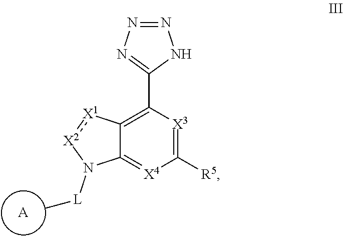

or a salt thereof wherein each variable is independently as described herein.

In some embodiments, the present disclosure provides a method, comprising:
reacting a compound having the structure of formula C1:

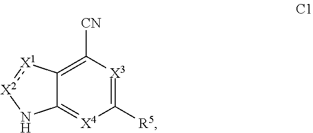

or a salt thereof with a compound having the structure of

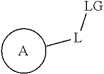

or a salt thereof, wherein LG is a leaving group to provide a compound having the structure of formula C3:

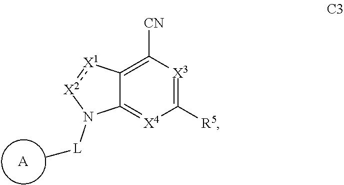

or a salt thereof, wherein each variable is independently as described herein.

In some embodiments, a method is described in Scheme 2 as an example.

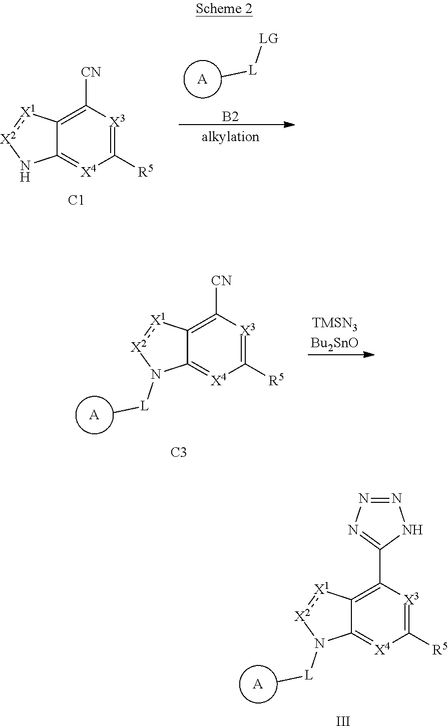

A method for preparing provided compounds, e.g., compounds of formula III or salts thereof, is illustrated in Scheme 2 as an example, wherein each variable is independently as described herein. In some embodiments, treatment of compound of formula C1 or a salt thereof, wherein X¹, X², X³, X⁴, and R⁵ are defined herein, and compound of formula B2 or a salt thereof, wherein ring A and L are defined herein and LG is a leaving group, under alkylation conditions such as Cs₂CO₃/DMF provides alkylated compound of formula C3 or a salt thereof. Various leaving groups may be utilized in accordance with the present disclosure. For example, in some embodiments, a leaving group is a halogen. In some embodiments, LG is Cl. In some embodiments, LG is Br. In some embodiments, LG is I. In some embodiments, LG is —OS(O)₂R wherein R is as described herein and is not H. In some embodiments, R is optionally substituted C₁₋₆ aliphatic. In some embodiments, R is optionally substituted phenyl. Many suitable conditions may be utilized in accordance with the present disclosure. For example, in some embodiments, a condition is an alkylation condition. In some embodiments, a reaction is performed in the presence of a base. In some embodiments, a base is Cs₂CO₃. Treatment of cyano compound of formula C3 or a salt thereof under tetrazole formation conditions such as TMSN₃/Bu₂SnO affords compound of formula III or a salt thereof.

In some embodiments, the present disclosure provides a method, comprising:
reacting a compound of formula D2

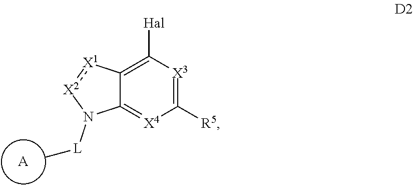

or a salt thereof to provide a compound having the structure of formula C3:

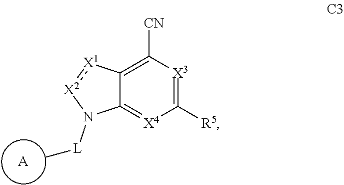

or a salt thereof wherein each variable is independently as described herein.

In some embodiments, the present disclosure provides a method, comprising:
reacting a compound having the structure of formula D1:

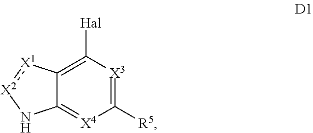

or a salt thereof with a compound having the structure of

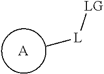

or a salt thereof, wherein LG is a leaving group to provide a compound having the structure of formula D2:

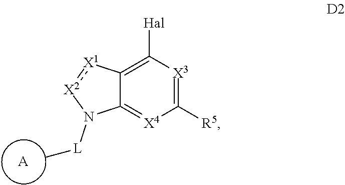

or a salt thereof, wherein each variable is independently as described herein.

In some embodiments, a method is described in Scheme 3 as an example:

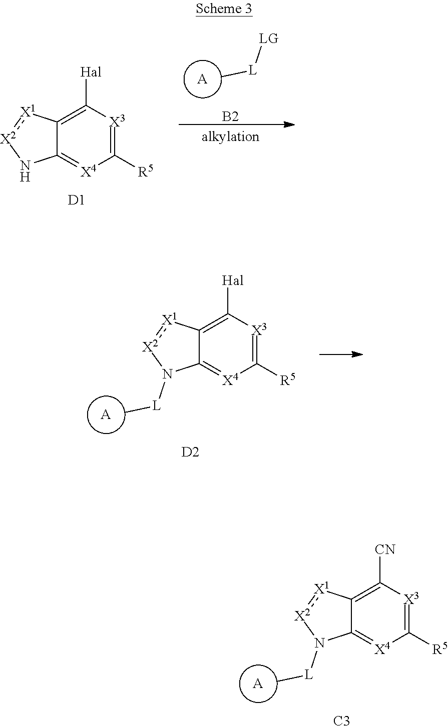

An alternative method for preparing provided compounds, e.g., compounds of formula C3 or salts thereof, is illustrated in Scheme 3 as an example, wherein each variable is independently as described herein. In some embodiments, treatment of compound of formula D1 or a salt thereof, wherein X¹, X², X³, X⁴, and R⁴ are defined herein and Hal is Cl, Br, or I, and compound of formula B2 or a salt thereof, wherein ring A and L are defined herein and LG is a leaving group, under alkylation conditions such as Cs₂CO₃/DMF provides alkylated compound of formula D2 or a salt thereof. Various leaving groups may be utilized in accordance with the present disclosure. For example, in some embodiments, a leaving group is a halogen. In some embodiments, LG is Cl. In some embodiments, LG is Br. In some embodiments, LG is I. In some embodiments, LG is —OS(O)₂R wherein R is as described herein and is not H. In some embodiments, R is optionally substituted C₁₋₆ aliphatic. In some embodiments, R is optionally substituted phenyl. Many suitable conditions may be utilized in accordance with the present disclosure. For example, in some embodiments, a condition is an alkylation condition. In some embodiments, a reaction is performed in the presence of a base. In some embodiments, a base is Cs₂CO₃. Treatment of compound of formula D2 or a salt thereof with cyanides such as CuCN or Zn(CN)₂ to replace Hal with CN affords compound of formula C3 or a salt thereof.

Certain technologies for preparing provided compounds are illustrated in the Examples.

### Use

In certain embodiments, the present disclosure provides a method for modulating MRGPRX4 activity by contacting MRGPRX4 with an effective amount of a compound or a pharmaceutical composition as described herein.

In certain embodiments, the present disclosure provides a method for modulating MRGPRX4 activity in a system comprising MRGPRX4, comprising administering or delivering to the system an effective amount of a compound or a pharmaceutical composition as described herein. In some embodiments, a system is or comprises a cell. In some embodiments, a system is or comprises a tissue. In some embodiments, a system is or comprises an organ. In some embodiments, a system is or comprises an organism. In some embodiments, a system is a subject. In some embodiments, a system is an animal. In some embodiments, a system is a human. In some embodiments, a system expresses MRGPRX4. In some embodiments, a method reduces MRGPRX4 activity level compared absence of a provided compound. In some embodiments, a reduction is about or at least about 20%, 25%, 30%, 40%, 50%, 60%, 70%, 80%, 90%, or 95%.

In certain embodiments, the present disclosure provides a method for preventing a condition, disorder or disease, comprising administering to a subject susceptible thereto an effective amount of a provided compound or composition. In certain embodiments, the present disclosure provides a method for treating a condition, disorder or disease, comprising administering to a subject suffering therefrom an effective amount of a provided compound or composition. In certain embodiments, the present disclosure provides a method for preventing a condition, disorder or disease, comprising delivering to a subject susceptible thereto an effective amount of a provided compound or composition. In certain embodiments, the present disclosure provides a method for treating a condition, disorder or disease, comprising delivering to a subject suffering therefrom an effective amount of a provided compound or composition. In some embodiments, a compound is administered or delivered as a pharmaceutically acceptable salt form. In some embodiments, a composition is a pharmaceutical composition. In some embodiments, to deliver a provided compound, a prodrug thereof may be administered.

In some embodiments, a compound is utilized in a racemic form. In some embodiments, a composition is a stereorandom mixture of multiple stereoisomers. For example, in some embodiments, a composition is a stereorandom mixture of two enantiomers. In some embodiments, a compound is utilized in a stereochemically pure form as described herein. In some embodiments, a compound is utilized in an enantiomerically pure form. In some embodiments, a composition is enriched for one or more stereoisomers over the others as described herein. In some embodiments, a composition is enriched for an enantiomer as described herein. In some embodiments, a composition is stereochemically pure. In some embodiments, a composition is enantiomerically pure.

In some embodiments, a condition, disorder or disease is or comprises itch. In some embodiments, a condition, disorder or disease is itch. In some embodiments, a condition, disorder or disease is a MRGPRX4-associated condition, disorder or disease. In some embodiments, a condition, disorder or disease is associated with MRGPRX4 activation.

In some embodiments, a condition, disorder or disease is chronic itch, cholestatic pruritus, contact dermatitis, allergic blepharitis, anemia, atopic dermatitis, bullous pemphigoid, candidiasis, chicken pox, cholestasis, end-stage renal failure, hemodialysis, contact dermatitis, dermatitis herpetiformis, diabetes, drug allergy, dry skin, dyshidrotic dermatitis, ectopic eczema, eczema, erythrasma, folliculitis, fungal skin infection, hemorrhoids, herpes, HIV infection, Hodgkin's disease, hyperthyroidism, iron deficiency anemia, kidney disease, leukemia, liver disease, lymphoma, malignancy, multiple myeloma, neurodermatitis, onchocerciasis, Paget's disease, pediculosis, polycythemia rubra vera, pruritus ani, pseudorabies, psoriasis, rectal prolapse, scabies, schistosomiasis, scleroderma, severe stress, stasis dermatitis, swimmer's itch, thyroid disease, tinea cruris, uremic pruritus, or urticaria.

In some embodiments, a condition, disorder or disease is MRGPRX4-associated pruritus. In some embodiments, a condition, disorder or disease is MRGPRX4-associated acute or chronic pruritus associated a liver condition, disorder or disease.

In certain embodiments, an MRGPRX4-associated pruritus is acute or chronic pruritus associated with a hepatobiliary condition, disorder or disease. In some embodiments, a hepatobiliary condition, disorder or disease is intrahepatic cholestasis of pregnancy (ICP), estrogen-, progesterone- or testosterone-induced cholestasis, toxin- or other drug induced hepatocellular cholestasis, benign recurrent intrahepatic cholestasis (BRIC), progressive familial intrahepatic cholestasis (PFIC), chronic viral hepatitis C, chronic hepatitis B, alcoholic or nonalcoholic fatty liver disease (NAFLD), nonalcoholic steatohepatitis (NASH), primary biliary cholangitis (PBC), primary sclerosing cholangitis (PSC), secondary sclerosing cholangitis (SSC), sarcoidosis, ABCB4 deficiency, alagille syndrome, drug-induce small duct cholangiopathies, gallstone disease, IgG4-associated cholangitis, biliary atresia, cholangiocellular carcinoma, benign bile duct adenoma, or other obstructive cholestasis.

In some embodiments, a condition, disorder or disease is a liver condition, disorder or disease. In some embodiments, a liver condition, disorder or disease is NASH. In some embodiments, a liver condition, disorder or disease is NAFLD. In some embodiments, a liver condition, disorder or disease is ICP. In some embodiments, a liver condition, disorder or disease is PBC. In some embodiments, a liver condition, disorder or disease is PFIC. In some embodiments, a liver condition, disorder or disease is PSC. In some embodiments, a liver condition, disorder or disease is BRIC. In some embodiments, a liver condition, disorder or disease is chronic hepatitis B.

In some embodiments, a condition, disorder or disease is nonalcoholic steatohepatitis (NASH). In some embodiments, a condition, disorder or disease is bile acid synthesis condition, disorder or disease. In some embodiments, a bile acid synthesis condition, disorder or disease is due to single enzyme defects (SEDs). In some embodiments, a condition, disorder or disease is a peroxisomal condition, disorder or disease, e.g., a Zellweger spectrum disorder. In some embodiments, a condition, disorder or disease is a liver condition, disorder or disease, steatorrhea or complications from decreased fat-soluble vitamin absorption.

In some embodiments, a condition, disorder or disease is cardiovascular disease, atherosclerosis, arteriosclerosis, hypercholesteremia, hyperlipidemia, chronic liver disease, gastrointestinal disease, renal disease, metabolic disease, cancer (i.e., colorectal cancer), or neurological indications such as stroke. In certain embodiments, a condition, disorder or disease is primary biliary cirrhosis (PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, or alpha 1-antitrypsin deficiency. In certain embodiments, a gastrointestinal disease is inflammatory bowel disease (IBD) (including Crohn's disease and ulcerative colitis), irritable bowel syndrome (IBS), bacterial overgrowth, malabsorption, postradiation colitis, or microscopic colitis. In certain embodiments, the renal disease is diabetic nephropathy, focal segmental glomerulosclerosis (FSGS), hypertensive nephrosclerosis, chronic glomerulonephritis, chronic transplant glomerulopathy, chronic interstitial nephritis, or poly cystic kidney disease. In certain embodiments, a cardiovascular disease is atherosclerosis, arteriosclerosis, dyslipidemia, hypercholesterolemia, or hypertriglyceridemia. In certain embodiments, a metabolic disease is insulin resistance, Type I and Type II diabetes, or obesity. In some embodiments, a condition, disorder or disease is an inflammatory condition, disorder or disease, e.g., allergy, osteoarthritis, appendicitis, bronchial asthma, pancreatitis, allergic rash, psoriasis, etc. In some embodiments, a condition, disorder or disease is an autoimmune condition, disorder or disease. In some embodiments, a condition, disorder or disease is rheumatoid arthritis, multiple sclerosis, and type I diabetes. In some embodiments, a condition, disorder or disease is a gastrointestinal disease, e.g., inflammatory bowel disease (Crohn's disease, ulcerative colitis), short bowel syndrome (post-radiation colitis), microscopic colitis, irritable bowel syndrome (malabsorption), and bacterial overgrowth. In some embodiments, a condition, disorder or disease is cancer. In some embodiments, a cancer is colorectal cancer, liver cancer, hepatocellular carcinoma, cholangiocarcinoma, renal cancer, gastric cancer, pancreatic cancer, prostate cancer, or insulanoma. In some embodiments, a condition, disorder or disease is FXR-mediated. In some embodiments, a condition, disorder or disease is TGR5-mediated. In some embodiments, subjects susceptible thereto and/or suffering therefrom benefit from increased levels of FXR and/or TGR5 activity. In some embodiments, a condition, disorder or disease is a chronic kidney disease. In some embodiments, a condition, disorder or disease is uremic pruritus.

In some embodiments, a condition, disorder or disease is associated with administration or delivery of an agent that can activate MRGPRX4. Many technologies for assessing MRGPRX4 activation, e.g., in vivo, in vitro, etc. can be utilized to assess if an agent can activate MRGPRX4. Certain useful technologies are described in, e.g., Meixiong et al. MRGPRX4 is a G protein-coupled receptor activated by bile acids or analogs or derivatives thereof that may contribute to cholestatic pruritus, PNAS, 2019, 116 (21), 10525-10530; Yu et al. MRGPRX4 is bile acid receptor for human cholestatic itch, eLife, 2019, 8, e48431.

In some embodiments, a condition, disorder or disease is associated with administration or delivery of an agent that can activate MRGPRX4 but can also provide another activity or can be utilized as a therapeutic agent for treating a condition, disorder or disease. In some embodiments, the present disclosure provides methods for preventing or treating a condition, disorder or disease associated with administration of an agent, comprising administering or delivering to a subject an effective amount of a provided compound or composition. In some embodiments, the present disclosure provides methods for preventing a condition, disorder or disease associated with administration of an agent, comprising administering to a subject an effective amount of a provided compound or composition. In some embodiments, the present disclosure provides methods for preventing a condition, disorder or disease associated with administration of an agent, comprising delivering to a subject an effective amount of a provided compound or composition. In some embodiments, the present disclosure provides methods for treating a condition, disorder or disease associated with administration of an agent, comprising administering to a subject an effective amount of a provided compound or composition. In some embodiments, the present disclosure provides methods for treating a condition, disorder or disease associated with administration of an agent, comprising delivering to a subject an effective amount of a provided compound or composition. In some embodiments, an agent can activate MRGPRX4. In some embodiments, an agent is a bile acid or a salt (e.g., a pharmaceutically acceptable salt) thereof. In some embodiments, an agent is a bile acid analog or a salt (e.g., a pharmaceutically acceptable salt) thereof. In some embodiments, an agent is a bile acid derivative or a salt (e.g., a pharmaceutically acceptable salt) thereof. In some embodiments, an agent is a bile acid conjugate, e.g., a taurine conjugate, or a salt (e.g., a pharmaceutically acceptable salt) thereof. In some embodiments, a bile acid is cholic acid. In some embodiments, a bile acid is ursodeoxycholic acid (UDCA). In some embodiments, a bile acid is ursocholic acid. In some embodiments, a bile acid is chenodeoxycholic acid. In some embodiments, a bile acid or an analog or derivative thereof is obeticholic acid. In some embodiments, a bile acid or an analog or derivative thereof is taurursodiol or a salt (e.g., a pharmaceutically acceptable salt) thereof; in some embodiments, it is taurursodiol; in some embodiments, it is sodium taurursodiol. In some embodiments, an agent is a FXR agonist. In some embodiments, an agent is a TGR5 agonist. In some embodiments, an agent is a therapeutic agent. In some embodiments, an agent is an approved therapeutic agent, e.g., by the U.S. Food and Drug Administration (e.g., cholic acid, obeticholic acid, taurursodiol, ursodeoxycholic acid, etc.), either individually or in combination with another therapeutic agent. In some embodiments, an agent is obeticholic acid and it is utilized in combination with ursodeoxycholic acid. In some embodiments, an agent is taurursodiol and it is utilized with sodium phenylbutyrate. In some embodiments, a condition, disorder or disease associated with administration of agent is or comprises itch. In some embodiments, provided methods can increase patient adherence of the agent. In some embodiments, provided methods can increase single doses, total doses, dose frequency, and/or length of dosage regimen of an agent. In some embodiments, provided compounds can reduce severity of a side effect, e.g., itch. In some embodiments, a provided compound is administered or delivered about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months before administration of an agent. In some embodiments, a provided compound is administered or delivered about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months after administration of an agent. In some embodiments, a provided compound is administered or delivered together with administration of an agent.

In certain embodiments, a compound as described herein can be used together with another therapeutic agent as a combination therapy to prevent or treat a condition, disorder or disease. In some embodiments, a condition, disorder or disease is associated with MRGPRX4 activation. In some embodiments, a therapeutic agent administered or delivered to a subject can activate MRGPRX4. In some embodiments, a provided compound can reduce a condition, disorder or disease associated with MRGPRX4 activation. In some embodiments, a condition, disorder or disease is or comprises concurring MRGPRX4 related pruritus. In certain embodiments, a therapeutic agent is a Farnesoid X receptor (FXR) agonist, such as obeticholic acid (OCA), cilofexor (GS-9674), tropifexor (LJN452), EDP-305, EDP-297, nidufexor, TERN-101 (LY2562175), MET-409, BAR704, BAR502, EYP-001, RDX-023, AGN-242266, HPG-1860, AGN-242256, IOT-022, M-480, INV-33, etc. In certain embodiments, a therapeutic agent is an ileal bile acid transport (IBAT) inhibitor, such as odevixibat, maralixibat, etc. In certain embodiments, a therapeutic agent is ursocholic acid. In certain embodiments, a therapeutic agent is ursodeoxycholic acid (UDCA). In certain embodiments, a therapeutic agent is a thyroid hormone receptor β (THR-β) agonist, such as resmetirom (MGL-3196), GC-24, MGL-3745, VK-2809, KB141 [3,5-dichloro-4-(4-hydroxy-3-isopropylphenoxy)phenylacetic acid], MB07811 (2R,4S)-4-(3-chlorophenyl)-2-((3,5-dimethyl-4-(4′-hydroxy-3′-isopropylbenzyl)phenoxy)methyl]-2-oxido-[1,3,2]-dioxaphosphonane), etc. In certain embodiments, a therapeutic agent is a peroxisome proliferator-activated receptors (PPAR) agonist, such as elafibranor, lanifibranor, saroglitazar, pioglitazone, rosiglitazone etc. In certain embodiments, a therapeutic agent is a glucagon-like peptide 1 (GLP-1) agonist, such as semaglutide, exenatide, dulaglutide, liraglutide, lixisenatide, danuglipron (PF-06882961) PF-07081532 etc. In certain embodiments, a therapeutic agent is a glucose-dependent insulinotropic polypeptide (GIP) receptor agonist such as tirzepatide. In certain embodiments, a therapeutic agent is an acetyl CoA-carboxylase (ACC) inhibitor such as firsocostat, PF-05221304, WZ66, etc. In certain embodiments, a therapeutic agent is a diacylglycerol O-acyltransferase 2 (DGAT2) inhibitor such as PF-06865571. In certain embodiments, the other therapeutic agent is a ketohexokinase (KHK) inhibitor such as PF-06835919. In some embodiments, a therapeutic agent is an approved agent (e.g., by U.S. Food and Drug Administration), either individually or as a combination, for treating a condition, disorder or disease. Various approved agents and their uses are publicly available and can be utilized with provided compounds in accordance with the present disclosure. For example, in some embodiments, an agent is obeticholic acid approved for treatment of adult patients with primary biliary cholangitis (PBC) without cirrhosis or with compensated cirrhosis who do not have evidence of portal hypertension, either in combination with ursodeoxycholic acid (UDCA) with an inadequate response to UDCA or as monotherapy in patients unable to tolerate UDCA. In some embodiments, an agent is cholic acid approved for treatment of bile acid synthesis disorders due to single enzyme defects (SEDs) or adjunctive treatment of peroxisomal disorders (PDs) including Zellweger spectrum disorders in patients who exhibit manifestations of liver disease, steatorrhea or complications from decreased fat-soluble vitamin absorption. In some embodiments, an agent is taurursodiol, in combination with sodium phenylbutyrate (Relyvrio) approved for the treatment of amyotrophic lateral sclerosis (ALS).

In some embodiments, a provided compound is administered or delivered concurrently with another therapeutic agent. In some embodiments, a provided compound is administered or delivered in a single composition with another therapeutic agent. In some embodiments, a provided compound is administered or delivered concurrently with another therapeutic agent but in different compositions. In some embodiments, a provided compound is administered or delivered prior to another therapeutic agent (e.g., about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months prior to another therapeutic agent). In some embodiments, a provided compound is administered or delivered after another therapeutic agent (e.g., about 1, 2, 3, 4, 5, 6, 7, 8, 9, 10, 11, 12, 13, 14, 15, 16, 17, 18, 19, or 20 days, or about 1, 2, 3, 4, or 5 weeks, or about 1, 2, 3, 4, or 5 months after another therapeutic agent). In some embodiments, a provided compound is administered or delivered when a subject is under the therapeutic effect of another therapeutic agent.

In some embodiments, a bile acid or an analog or derivative thereof is reported in Meixiong et al. MRGPRX4 is a G protein-coupled receptor activated by bile acids that may contribute to cholestatic pruritus, PNAS, 2019, 116 (21), 10525-10530; Yu et al. MRGPRX4 is bile acid receptor for human cholestatic itch, eLife, 2019, 8, e48431. In some embodiments, a bile acid or an analog or derivative thereof is reported in WO 2016086169, U.S. Pat. No. 10,519,191, WO 2016130809, U.S. Pat. No. 10,246,483, WO 2017147174, WO 2017147159, WO 2017147137, U.S. Pat. Nos. 10,364,267, 10,323,061, 10,323,060, WO 2017189663, WO 2017189652, WO 2017189651, U.S. Pat. Nos. 10,080,743, 10,080,742, 10,080,741, WO 2017201155, WO 2017201152, WO 2017201150, U.S. Pat. Nos. 10,144,729, 10,138,228, WO 2018067704, U.S. Pat. No. 10,450,306, WO 2018081285, U.S. Pat. No. 10,597,391, WO 2018102418, U.S. Pat. No. 10,584,145, WO 2018152171, U.S. Pat. No. 10,472,386, WO 2018187804, U.S. Pat. No. 10,676,500, WO 2019118571, U.S. Pat. No. 10,689,391, WO 2020231917, WO 2016073767, U.S. Pat. No. 10,266,560, WO 2016086134, U.S. Pat. No. 10,208,081, WO 2016086218, U.S. Pat. Nos. 10,696,713, 10,968,249, WO 2016086115, WO 2016161003, U.S. Pat. Nos. 10,457,703, 11,040,998, 11,034,684, 10,947,264, 10,961,272, WO 2019160813, or U.S. Pat. No. 10,829,486, the bile acids and analogs or derivatives thereof of each of which are incorporated herein by reference. In some embodiments, a FXR agonist is reported in WO 2016086169, U.S. Pat. No. 10,519,191, WO 2016130809, U.S. Pat. No. 10,246,483, WO 2017147174, WO 2017147159, WO 2017147137, U.S. Pat. Nos. 10,364,267, 10,323,061, 10,323,060, WO 2017189663, WO 2017189652, WO 2017189651, U.S. Pat. Nos. 10,080,743, 10,080,742, 10,080,741, WO 2017201155, WO 2017201152, WO 2017201150, U.S. Pat. No. 10,144,729, U.S. Ser. No. 10/138,228, WO 2018067704, U.S. Pat. No. 10,450,306, WO 2018081285, U.S. Pat. No. 10,597,391, WO 2018102418, U.S. Pat. No. 10,584,145, WO 2018152171, U.S. Pat. No. 10,472,386, WO 2018187804, U.S. Pat. No. 10,676,500, WO 2019118571, U.S. Pat. No. 10,689,391, WO 2020231917, WO 2016073767, U.S. Pat. No. 10,266,560, WO 2016086134, U.S. Pat. No. 10,208,081, WO 2016086218, U.S. Pat. Nos. 10,696,713, 10,968,249, WO 2016086115, WO 2016161003, U.S. Pat. Nos. 10,457,703, 11,040,998, 11,034,684, 10,947,264, 10,961,272, WO 2019160813, or U.S. Pat. No. 10,829,486, the FXR agonists of each of which are incorporated herein by reference.

In some embodiments, a condition, disorder or disease is primary biliary cholangitis (PBC) without cirrhosis or with compensated cirrhosis who do not have evidence of portal hypertension. In some embodiments, another therapeutic agent is obeticholic acid. In some embodiments, it is in combination with ursodeoxycholic acid (UDCA).

In some embodiments, a condition, disorder or disease is a bile acid synthesis disorder. In some embodiments, a condition, disorder or disease is a bile acid synthesis disorder due to single enzyme defects (SEDs). In some embodiments, a condition, disorder or disease is peroxisomal disorders (PDs). In some embodiments, a condition, disorder or disease is peroxisomal disorders (PDs) including Zellweger spectrum disorders. In some embodiments, a subject exhibits manifestations of liver disease, steatorrhea or complications. In some embodiments, another therapeutic agent is cholic acid.

In some embodiments, a condition, disorder or disease is a neurodegenerative condition, disorder or disease. In some embodiments, a condition, disorder or disease is ALS. In some embodiments, another therapeutic agent is taurursodiol in combination with sodium phenylbutyrate (Relyvrio).

In some embodiments, a subject is an adult patient. In some embodiments, a subject is a pediatric patient.

### Pharmaceutical Compositions and Administration

In some embodiments, the present disclosure provides a pharmaceutical composition that comprise a provided compound or a pharmaceutically acceptable salt thereof, and a pharmaceutically acceptable carrier. In some embodiments, the present disclosure provides a pharmaceutical composition that can deliver a provided compound or a pharmaceutically acceptable salt thereof, and a pharmaceutically acceptable carrier.

Various technologies, e.g., routes, modes, dosage regimens, etc. may be utilized to administer and/or deliver provided compounds and compositions in accordance with the present disclosure. In some embodiments, a route and/or mode of administration can vary depending upon desired results. One with skill in the art, i.e., a physician, is aware that dosage regimens can be adjusted to provide a desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. In some embodiments, a mode of administration is left to discretion of a practitioner.

In some embodiments, compounds can be incorporated into and administered as pharmaceutical compositions. Such pharmaceutical compositions are useful for, among other things, administration and delivery to a subject in vivo or ex vivo. In some embodiments, pharmaceutical compositions also contain a pharmaceutically acceptable carrier. In some embodiments, a pharmaceutically acceptable carrier is a pharmaceutical agent that does not itself induce an immune response harmful to the individual receiving a composition, and which may be administered without undue toxicity. Pharmaceutically acceptable carriers (or excipients) include, but are not limited to, liquids such as water, saline, glycerol, sugars and ethanol. Additionally, auxiliary substances, such as wetting or emulsifying agents, pH buffering substances, and the like, may be present in such vehicles.

Compounds in pharmaceutical compositions may be provided as pharmaceutically acceptable salts. In some embodiments, salts can be formed with many acids, including but not limited to, hydrochloric, sulfuric, acetic, lactic, tartaric, malic, succinic, benzenesulfonic, etc. In some embodiments, salts can be formed with bases. In some embodiments, salts are alkali, alkaline earth metal, or ammonium salts, e.g., sodium, calcium, diethanolamine, ethanolamine, trialkylamine salts, etc.

In some embodiments, salts are more soluble in aqueous or other protonic solvents than corresponding, free acid or base forms. In some embodiments, a pharmaceutical composition may be a lyophilized powder. In some embodiments, a pharmaceutical composition comprises a provided compound, e.g., a compound of formula I or a pharmaceutically acceptable salt thereof dissolved in a pharmaceutically acceptable buffer. In some embodiments, a buffer is a saline buffer. In some embodiments, a buffer has a pH around 7.4.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. In some embodiments, pharmaceutical compositions or formulations are tablets (coated or uncoated), capsules (hard or soft), microbeads, powder, granules and/or crystals. Supplementary active compounds (e.g., preservatives, antibacterial, antiviral and antifungal agents) can also be incorporated into pharmaceutical compositions.

Pharmaceutical compositions can be formulated to be compatible with a particular route of administration or delivery as set forth herein or known to one of skill in the art.

In some embodiments, provided compositions are suitable for parenteral administration. In some embodiments, such compositions comprise aqueous and non-aqueous solutions, suspensions or emulsions of active compounds, which preparations are typically sterile and can be isotonic with blood of intended recipients. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, a suspension may also contain suitable stabilizers or agents which increase solubility to allow for the preparation of highly concentrated solutions.

Co-solvents and adjuvants may be added to compositions and formulations. Non-limiting examples of co-solvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Various pharmaceutical compositions and delivery systems appropriate for compositions, methods and uses of the present disclosure are known in the art (see, e.g., Remington: The Science and Practice of Pharmacy. 21 st Edition. Philadelphia, PA. Lippincott Williams & Wilkins, 2005) and can be utilized in accordance with the present disclosure.

In some embodiments, the present disclosure provides methods for delivering provided compounds and compositions into cells, animals or subjects. In some embodiments, such methods include contacting a subject (e.g., a cell or tissue of a subject) with, or administering or delivering to a subject (e.g., a subject such as a mammal or human) a provided compound, e.g., a compound of formula I or a salt thereof, or a composition thereof.

A compound or composition described herein can be administered in a sufficient or effective amount to a subject (or a cell, tissue or organ thereof) in need thereof. Doses can vary and may depend upon the type, onset, progression, severity, frequency, duration, or probability of a condition, disorder or disease to which treatment is directed, a clinical endpoint desired, previous or simultaneous treatments, general health, age, gender, race or immunological competency of a subject and other factors that will be appreciated by a skilled artisan. Dose amount, number, frequency or duration may be proportionally increased or reduced, as indicated by efficacy, any adverse side effects, complications or other risk factors of a treatment or therapy and the status of a subject. A skilled artisan will appreciate factors that may influence dosage and timing required to provide an amount sufficient for providing a therapeutic or prophylactic benefit.

A dose to achieve a therapeutic effect will vary based on several factors including route of administration, amount to achieve a therapeutic effect, specific condition, disorder or disease treated, any host immune response to administered compound or composition, stability of administered compound or composition, etc.

An effective amount or a sufficient amount can be provided in a single administration, may require multiple administrations, and, can be, administered alone or in combination with another composition (e.g., comprising or delivering another therapeutic agent). For example, an amount may be proportionally increased as indicated by the need of a subject, type, status and severity of a condition, disorder or disease treated and/or side effects (if any) of treatment. Amounts considered effective also include amounts that result in a reduction of the use of another treatment, therapeutic regimen or protocol.

In some embodiments, pharmaceutical compositions comprise or deliver active ingredients, e.g., compounds of formula I or pharmaceutically acceptable salts thereof, in effective amounts to achieve intended purposes e.g., therapeutic purposes. Various technologies may be utilized to determine therapeutically effective amounts in accordance with the present disclosure. Therapeutic doses can depend on, among other factors, ages and general conditions of subjects, severity of conditions, disorders or diseases, etc. In some embodiments, therapeutically effective amounts in humans may fall in a relatively broad range that may be determined by medical practitioners based on responses of individual patients.

In some embodiments, methods and uses of the present disclosure include delivery and administration systemically, regionally or locally, or by any route, for example, by injection or infusion or orally. In some embodiments, delivery of a pharmaceutical composition in vivo may generally be accomplished via injection using a conventional syringe, although other delivery methods such as convection-enhanced delivery can also be used In some embodiments, compounds and compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. In some embodiments, modes of administration include oral and pulmonary administration, suppositories, and transdermal applications. Clinicians specializing in treating patients may determine optimal routes for administration of compounds and compositions as described herein.

### EXEMPLIFICATION

Certain examples of provided technologies (e.g., compounds, compositions, methods (methods of preparation, use, assessment, etc.), etc.) are described herein. Those skilled in the art reading the present disclosure appreciate that various technologies, including those described below and modifications, variants and derivatives thereof, are available for manufacturing, characterizing and/or assessing provided technologies in accordance with the present disclosure.

### Example 1. Compounds 1-37

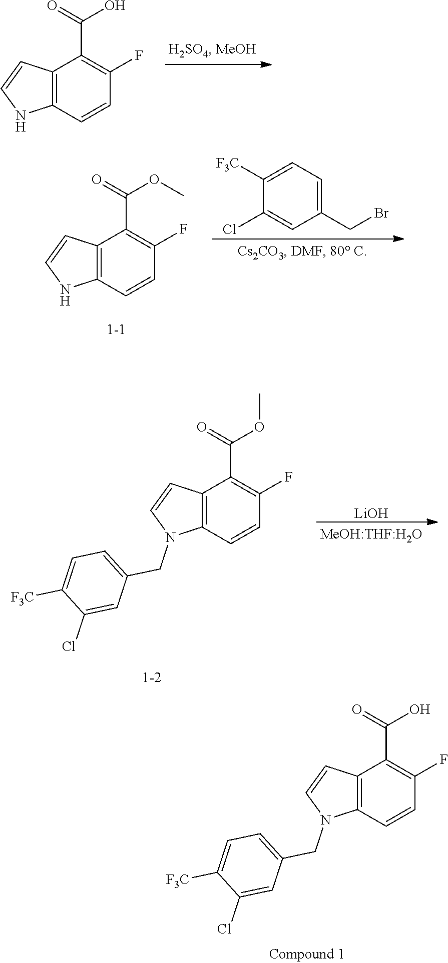 [SMILES: O=C(O)c1c(F)ccc2[nH]ccc12 ; COC(=O)c1c(F)ccc2[nH]ccc12 ; FC(F)(F)c1ccc(CBr)cc1Cl ; COC(=O)c1c(F)ccc2c1ccn2Cc1ccc(C(F)(F)F)c(Cl)c1 ; "LiOH" ; O=C(O)c1c(F)ccc2c1ccn2Cc1ccc(C(F)(F)F)c(Cl)c1]

Step 1: To a solution of 5-fluoro-1H-indole-4-carboxylic acid (250 mg, 1.40 mmol) dissolved in MeOH (5 mL) was added H₂SO₄ (100 μL) dropwise. The resulting solution was stirred at 80° C. for 2 h. After complete conversion, the reaction mixture was cooled to room temperature, quenched with water, and extracted with EA for three times. The combined organic layer was washed with brine, dried over anhydrous Na₂SO₄, filtered, and concentrated in vacuo. Purification of the residue via column chromatography afforded 1-1 (264 mg, 62% yield) as colorless oil. MS (m/z): 194.03 (M+1).

Step 2: Compound 1-1 (100 mg, 0.518 mmol), 4-(bromomethyl)-2-chloro-1-(trifluoromethyl)benzene (169 mg, 0.622 mmol) and Cs₂CO₃ (506 mg, 1.554 mmol) were added to DMF (3 mL) and the resulting mixture was heated at 80° C. for 2 h. After complete conversion, the reaction mixture was cooled to room temperature, quenched with water, and extracted with EA for three times. The combined organic layer was washed with brine, dried over anhydrous Na₂SO₄, filtered, and concentrated in vacuo. Purification of the residue via column chromatography afforded compound 1-2 (50.8 mg, 26% yield) as yellow solid. MS (m/z): 386.2 (M+1).

Step 3: Compound 1-2 (50.8 mg, 0.132 mmol) and LiOH·H₂O (31.7 mg, 1.319 mmol) were dissolved in THF/MeOH/H₂O (1:1:1, 2 mL) under Ar. The mixture was stirred at room temperature for 2 h. After complete conversion, the solution was adjusted to pH 6 with aq. HCl and concentrated. The residue was purified by reversed phase high-performance liquid chromatography to afford Compound 1 (9 mg, 18.4% yield) as white solid. LC-MS, ES (m/z): 372.08 (M+1). 1H NMR (400 MHz, Methanol-d4) δ 7.78 (d, J=1.9 Hz, 1H), 7.51-7.41 (m, 3H), 7.04 (dd, J=3.2, 0.9 Hz, 1H), 6.97 (dd, J=11.2, 8.9 Hz, 1H), 6.69 (d, J=8.1 Hz, 1H), 5.61 (s, 2H).

The following examples were prepared employing the same protocol as described for Compound 1.

| Compound | Structure | NMR | MS |
|---|---|---|---|
| 2 | 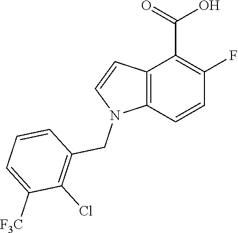 [SMILES: O=C(O)c1c(F)ccc2c1ccn2Cc1cccc(C(F)(F)F)c1Cl] | ¹H NMR (400 MHz, Methanol-d4) δ 7.71 (d, J = 7.9 Hz, 1H), 7.52-7.45 (m, 2H), 7.33 (t, J = 7.8 Hz, 1H), 7.05 (dd, J = 3.2, 0.9 Hz, 1H), 6.98 (dd, J = 11.3, 8.9 Hz, 1H), 6.72 (d, J = 7.8 Hz, 1H), 5.65 (s, 2H). | 372.03 (M + 1) |
| 3 | 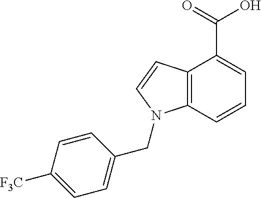 [SMILES: O=C(O)c1cccc2c1ccn2Cc1ccc(C(F)(F)F)cc1] | ¹H NMR (400 MHz, Chloroform-d) δ 8.05-8.03 (m, 1H), 7.57-7.55 (m, 2H), 7.48-7.46 (m, 1H), 7.33- 7.31 (m, 2H), 7.28-7.24 (m, 1H), 7.18-7.16 (m, 2H), 5.46(s, 2H). | 320.01 (M + 1) |
| 4 | 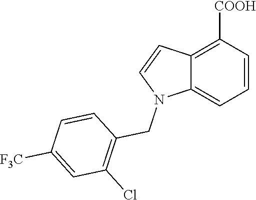 [SMILES: O=C(O)c1cccc2c1ccn2Cc1ccc(C(F)(F)F)cc1Cl] | ¹H NMR (400 MHz, DMSO-d₆) δ 7.95 (d, J = 1.9 Hz, 1H), 7.76 (dd, J = 7.4, 0.9 Hz, 1H), 7.67 (dd, J = 8.2, 1.0 Hz, 1H), 7.64-7.58 (m, 2H), 7.24-7.19 (m, 1H), 7.08 (d, J = 3.1 Hz, 1H), 6.64 (d, J = 8.1 Hz, 1H), 5.68 (s, 2H). | 352 (M − H)⁻ |
| 5 | 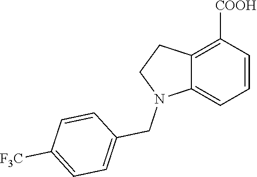 [SMILES: O=C(O)c1cccc2c1CCN2Cc1ccc(C(F)(F)F)cc1] | ¹H NMR (400 MHz, DMSO-d₆) δ 7.73 (d, J = 8.0 Hz, 2H), 7.58 (d, J = 8.0 Hz, 2H), 7.17-7.07 (m, 2H), 6.75 (dd, J = 7.6, 1.2 Hz, 1H), 4.44 (s, 2H), 3.33 (s, 2H), 3.26 (dd, J = 9.8, 7.7 Hz, 3H). | 320 (M − H)⁻ |
| 6 | 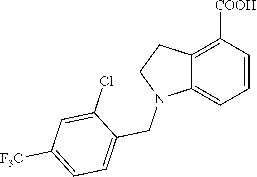 [SMILES: O=C(O)c1cccc2c1CCN2Cc1ccc(C(F)(F)F)cc1Cl] | ¹H NMR (400 MHz, DMSO-d₆) δ 7.93 (s, 1H), 7.75 (d, J = 8.2 Hz, 1H), 7.66 (d, J = 8.1 Hz, 1H), 7.18 (dd, J = 7.8, 1.1 Hz, 1H), 7.11 (t, J = 7.7 Hz, 1H), 6.72-6.67 (m, 1H), 4.48 (s, 2H), 3.46 (d, J = 8.7 Hz, 2H), 3.29 (d, J = 8.5 Hz, 2H). | 354 (M − H)⁻ |
| 7 | 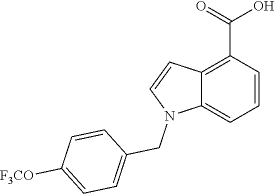 [SMILES: O=C(O)c1cccc2c1ccn2Cc1ccc(OC(F)(F)F)cc1] | ¹H NMR (400 MHz, Chloroform-d) δ 8.03 (d, J = 8.2 Hz, 1H), 7.50 (d, J = 8.2 Hz, 1H), 7.33-7.28 (m, 2H), 7.26 (d, J = 2.2 Hz, 1H), 7.16 (d, J = 8.7 Hz, 2H), 7.10 (d, J = 8.8 Hz, 2H), 5.40 (s, 2H). | 335.93 (M + 1) |
| 8 | 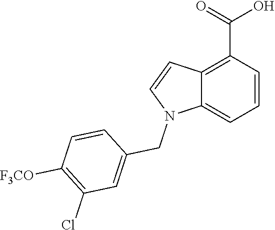 [SMILES: O=C(O)c1cccc2c1ccn2Cc1ccc(OC(F)(F)F)c(Cl)c1] | ¹H NMR (400 MHz, Chloroform-d) δ 8.05 (d, J = 7.4 Hz, 1H), 7.49 (d, J = 8.1 Hz, 1H), 7.35-7.27 (m, 3H), 7.23 (dd, J = 4.7, 1.7 Hz, 2H), 6.95 (dd, J = 8.5, 2.2 Hz, 1H), 5.38 (s, 2H). | 370.01 (M + 1) |
| 9 | 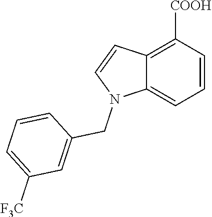 [SMILES: O=C(O)c1cccc2c1ccn2Cc1cccc(C(F)(F)F)c1] | ¹H NMR (400 MHz, DMSO-d₆) δ 12.66 (s, 1H), 7.83-7.71 (m, 3H), 7.64 (d, 2H), 7.61-7.51 (m, 1H), 7.44 (d, 1H), 7.22 (dd, 1H), 7.05 (dd,1H), 5.63 (s, 2H). | 320.03 (M + 1) |
| 10 | 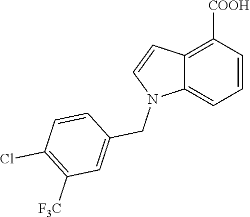 [SMILES: O=C(O)c1cccc2c1ccn2Cc1ccc(Cl)c(C(F)(F)F)c1] | ¹H NMR (400 MHz, DMSO-d₆) δ 12.70 (s, 1H), 7.82-7.72 (m, 4H), 7.68 (d, 1H), 7.39 (dd, 1H), 7.23 (t, 1H), 7.06 (d, 1H), 5.62 (s, 2H) | 353.96 (M + 1) |
| 11 | 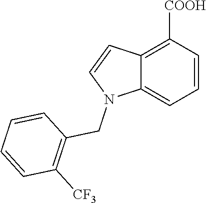 [SMILES: O=C(O)c1cccc2c1ccn2Cc1ccccc1C(F)(F)F] | ¹H NMR (400 MHz, DMSO-d₆) δ 12.63 (s, 1H), 7.83 (dd,1H), 7.78 (dd, 1H), 7.64 (d, 1H), 7.51 (td, 3H), 7.22 (t, 1H), 7.11 (dd, 1H), 6.53-6.41 (m, 1H), 5.72 (s, 2H). | 320.03 (M + 1) |
| 12 | 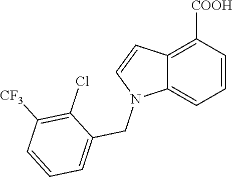 [SMILES: O=C(O)c1cccc2c1ccn2Cc1cccc(C(F)(F)F)c1Cl] | ¹H NMR (400 MHz, Methanol-d4) δ 7.88 (d, J = 7.6 Hz, 1H), 7.72 (d, J = 7.6 Hz, 1H), 7.54 (d, J = 8.0 Hz, 1H), 7.47 (d, J = 3.2 Hz, 1H), 7.33 (t, J = 8.0 Hz, 1H), 7.26-7.22 (m, 2H), 6.70 (d, J = 7.6 Hz, 1H), 5.69 (s, 2H). | 353.97 (M + 1) |
| 13 | 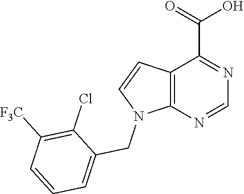 [SMILES: O=C(O)c1ncnc2c1ccn2Cc1cccc(C(F)(F)F)c1Cl] | ¹H NMR (400 MHz, DMSO-d₆) δ 8.92 (s, 1H), 7.88 (d, J = 3.6 Hz, 1H), 7.81 (d, J = 7.8 Hz, 1H), 7.45 (t, J = 7.8 Hz, 1H), 7.09-6.94 (m, 2H), 5.72 (s, 2H). | 354.25 (M − 1) |
| 14 | 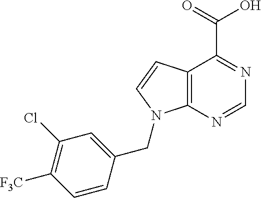 [SMILES: O=C(O)c1ncnc2c1ccn2Cc1ccc(C(F)(F)F)c(Cl)c1] | ¹H NMR (400 MHz, DMSO-d₆) δ 8.93 (s, 1H), 8.00-7.87 (m, 2H), 7.63 (d, J = 9.1 Hz, 1H), 7.03 (d, J = 3.5 Hz, 1H), 6.94 (d, J = 8.1 Hz, 1H), 5.71 (s, 2H). | 354.19 (M − 1) |
| 15 | 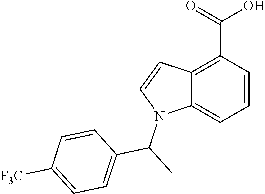 [SMILES: CC(c1ccc(C(F)(F)F)cc1)n1ccc2c(C(=O)O)cccc21] | 1H NMR (400 MHz, Chloroform-d) δ 7.78 (dd, J = 7.5, 0.9 Hz, 1H), 7.50 (dd, J = 7.4, 1.0 Hz, 1H), 7.30 (dd, J = 8.1, 5.3 Hz, 2H), 7.26 (d, J = 3.4 Hz, 1H), 7.19-7.08 (m, 1H), 7.01-6.90 (m, 3H), 5.50 (dd, J = 12.6, 6.8 Hz, 1H), 1.72 (dd, J = 14.0, 7.1 Hz, 3H). | 333.99 (M + 1) |
| 16 | 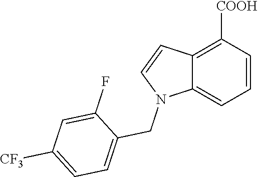 [SMILES: O=C(O)c1cccc2c1ccn2Cc1ccc(C(F)(F)F)cc1F] | ¹H NMR (400 MHz, Methanol-d4) δ 7.89-7.87 (m, 1H), 7.631 (d, J = 8 Hz, 1H), 7.54-7.949 (m, 2H), 7.387 (d, J = 8.4 Hz, 1H), 7.246 (t, J = 8.0 Hz, 1H), 7.20-7.19 (m, 1H), 6.996 (t, J = 7.6 Hz, 1H), 5.623 (s, 2H). | 336.25 (M − 1) |
| 17 | 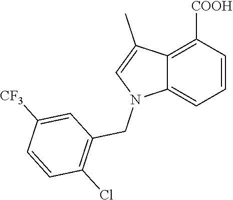 [SMILES: Cc1cn(Cc2cc(C(F)(F)F)ccc2Cl)c2cccc(C(=O)O)c12] | ¹H NMR (400 MHz, DMSO-d6) δ 12.75 (s, 1H), 7.80 (d, J = 8.4 Hz, 1H), 7.74-7.72 (m, 1H), 7.64- 7.62 (m, 1H), 7.49-7.47 (m, 1H), 7.36 (s, 1H), 7.18 (t, J = 7.2 Hz, 1H), 7.06 (d, J = 7.6 Hz, 1H), 5.59 (s, 2H), 2.35 (s, 3H). | 368.01 (M + 1) |
| 18 | 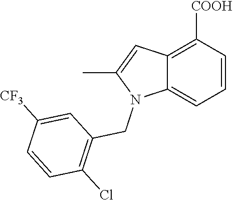 [SMILES: Cc1cc2c(C(=O)O)cccc2n1Cc1cc(C(F)(F)F)ccc1Cl] | ¹H NMR (400 MHz, DMSO-d6) δ 7.84(d, J = 8.4 Hz, 1H), 7.72 (t, J = 8.0 Hz, 2H), 7.59 (d, J = 8.0 Hz, 1H), 7.13 (t, J = 8.0 Hz, 1H), 6.99 (s, 1H), 6.37 (s, 1H), 5.61(s, 2H), 2.37 (s, 3H). | 368.03 (M + 1) |
| 19 | 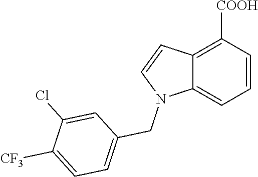 [SMILES: O=C(O)c1cccc2c1ccn2Cc1ccc(C(F)(F)F)c(Cl)c1] | ¹H NMR (400 MHz, Methanol-d4) δ 7.89 (d, J = 7.2 Hz, 1H), 7.82 (s, 1H), 7.53 (d, J = 8.4 Hz, 1H), 7.48-7.44 (m, 2H), 7.25-7.21 (m, 2H), 6.66 (d, J = 8.4 Hz, 1H), 5.65 (s, 2H). | 354.01 (M + 1) |
| 20 | 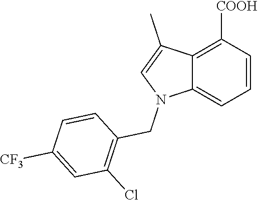 [SMILES: Cc1cn(Cc2ccc(C(F)(F)F)cc2Cl)c2cccc(C(=O)O)c12] | ¹H NMR (400 MHz, Methanol-d4) δ 7.83 (s, 1H), 7.62 (d, J = 7.2 Hz, IH), 7.47-7.42 (m, 2H), 7.23- 7.18 (m, 2H), 6.67 (d, J = 8.0 Hz, 1H), 5.57 (s, 2H), 2.48 (s, 3H). | 368.04 (M + 1) |
| 21 | 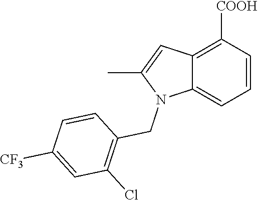 [SMILES: Cc1cc2c(C(=O)O)cccc2n1Cc1ccc(C(F)(F)F)cc1Cl] | ¹H NMR (400 MHz, DMSO-d6) δ 12.46 (s, 1H), 7.97 (s, 1H), 7.73-7.71 (m, 1H), 7.59-7.53 (m, 2H), 7.12 (t, J = 8.0 Hz, 1H), 6.94 (s, 1H), 6.25 (d, J = 8.0 Hz, 1H), 5.60 (s, 2H), 2.36 (s, 3H). | 368.02 (M + 1) |
| 22 | 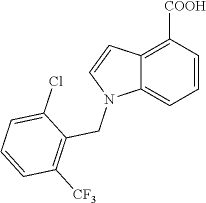 [SMILES: O=C(O)c1cccc2c1ccn2Cc1c(Cl)cccc1C(F)(F)F] | ¹H NMR (400 MHz, CDCl₃) δ 8.04 (d, J = 8 Hz, 1H), 7.76 (d, J = 8 Hz, 2H), 7.69 (d, J = 8 Hz, 1H), 7.53 (m, 1H), 7.35 (m, 1H), 7.14 (d, J = 4 Hz, 1H), 6.80 (d, J = 4 Hz, 1H), 5.54 (s, 2H). | 354.02 (M + 1) |
| 23 | 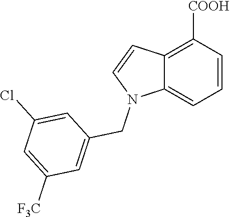 [SMILES: O=C(O)c1cccc2c1ccn2Cc1cc(Cl)cc(C(F)(F)F)c1] | ¹H NMR (400 MHz, CDCl₃) δ 8.06 (d, J = 8 Hz, 1H), 7.54 (s, 1H), 7.45 (d, J = 8 Hz, 1H), 7.32 (m, 4H), 7.17 (s, 1H), 5.43 (s, 2H). | 353.98 (M + 1) |
| 24 | 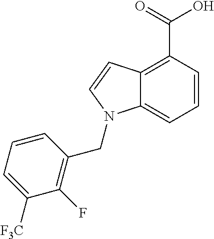 [SMILES: O=C(O)c1cccc2c1ccn2Cc1cccc(C(F)(F)F)c1F] | ¹H NMR (400 MHz, Chloroform-d) δ 8.04 (d, J = 8.3 Hz, 1H), 7.53 (t, J = 6.6 Hz, 2H), 7.35 (d, J = 3.2 Hz, 1H), 7.33-7.27 (m, 2H), 7.10 (t, J = 7.7 Hz, 1H), 6.90 (t, J = 8.0 Hz, 1H), 5.50 (s, 2H). | 337.98 (M + 1) |
| 25 | 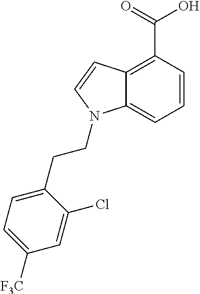 [SMILES: O=C(O)c1cccc2c1ccn2CCc1ccc(C(F)(F)F)cc1Cl] | ¹H NMR (400 MHz, Chloroform-d) δ 8.03 (dd, J = 7.5, 0.9 Hz, 1H), 7.72-7.67 (m, 1H), 7.58 (d, J = 8.1 Hz, 1H), 7.36-7.32 (m, 1H), 7.32-7.26 (m, 1H), 7.19 (dd, J = 3.2, 0.8 Hz, 1H), 7.13 (d, J = 3.2 Hz, 1H), 7.00 (d, J = 8.0 Hz, 1H), 4.50 (t, J = 7.1 Hz, 2H), 3.34 (t, J = 7.1 Hz, 2H). | 368.04 (M + 1) |
| 26 | 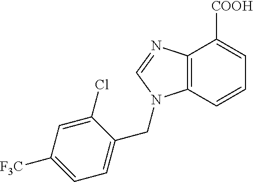 [SMILES: O=C(O)c1cccc2c1ncn2Cc1ccc(C(F)(F)F)cc1Cl] | ¹H NMR (400 MHz, Methanol-d₄) δ 8.60 (s, 1H), 8.06 (d, J = 7.5 Hz, 1H), 7.91 (s, 1H), 7.78 (d, J = 8.1 Hz, 1H), 7.66 (d, J = 8.0 Hz, 1H), 7.49 (t, J = 7.9 Hz, 1H), 7.25 (d, J = 8.1 Hz, 1H), 5.87 (s, 2H). | 355.01 (M + 1) |
| 27 | 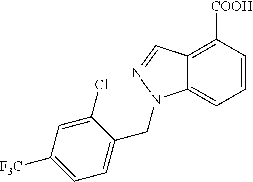 [SMILES: O=C(O)c1cccc2c1cnn2Cc1ccc(C(F)(F)F)cc1Cl] | ¹H NMR (400 MHz, DMSO-d₆) δ 13.23 (s, 1H), 8.49 (d, J = 0.9 Hz, 1H), 8.05 (d, J = 8.4 Hz, 1H), 7.94 (d, J = 1.8 Hz, 1H), 7.86 (d, J = 7.1 Hz, 1H), 7.65 (dd, J = 8.2, 1.9 Hz, 1H), 7.55 (dd, J = 8.4, 7.2 Hz, 1H), 6.95 (d, J = 8.1 Hz, 1H), 5.91 (s, 2H). | 355.03 (M + 1) |
| 28 | 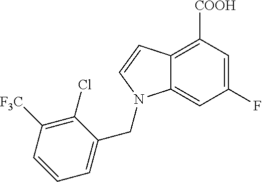 [SMILES: O=C(O)c1cc(F)cc2c1ccn2Cc1cccc(C(F)(F)F)c1Cl] | ¹H NMR (400 MHz, DMSO-d₆) δ 13.09 (s, 1H), 7.81 (dd, J = 8.0, 1.6 Hz, 1H), 7.76 (dd, J = 9.7, 2.4 Hz, 1H), 7.63 (d, J = 3.3 Hz, 1H), 7.53 (dd, J = 10.1, 2.4 Hz, 1H), 7.45 (t, J = 7.8 Hz, 1H), 7.09 (d, J = 3.2 Hz, 1H), 6.71 (d, J = 7.8 Hz, 1H), 5.69 (s, 2H). | 371.90 (M + 1) |
| 29 | 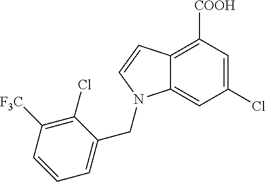 [SMILES: O=C(O)c1cc(Cl)cc2c1ccn2Cc1cccc(C(F)(F)F)c1Cl] | ¹H NMR (400 MHz, DMSO-d₆) δ 13.12 (s, 1H), 7.99 (d, J = 1.9 Hz, 1H), 7.82 (dd, J = 7.9, 1.6 Hz, 1H), 7.71 (d, J = 1.9 Hz, 1H), 7.67 (d, J = 3.2 Hz, 1H), 7.45 (t, J = 7.9 Hz, 1H), 7.10 (d, J = 3.1 Hz, 1H), 6.66 (d, J = 7.7 Hz, 1H), 5.73 (s, 2H). | 387.94 (M + 1) |
| 30 | 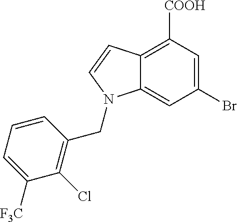 [SMILES: O=C(O)c1cc(Br)cc2c1ccn2Cc1cccc(C(F)(F)F)c1Cl] | ¹H NMR (400 MHz, DMSO-d₆) δ 13.12 (s, 1H), 8.12 (dd, J = 1.7, 0.9 Hz, 1H), 7.82 (dd, J = 10.5, 1.6 Hz, 2H), 7.66 (d, J = 3.2 Hz, 1H), 7.45 (t, J = 7.8 Hz, 1H), 7.10 (dd, J = 3.2, 0.8 Hz, 1H), 6.67- 6.61 (m, 1H), 5.73 (s, 2H). | 429.95 (M − 1). |
| 31 | 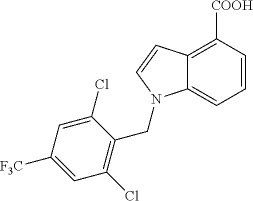 [SMILES: O=C(O)c1cccc2c1ccn2Cc1c(Cl)cc(C(F)(F)F)cc1Cl] | ¹H NMR (400 MHz, DMSO-d₆) δ 12.69 (s, 1H), 8.09 (s, 2H), 7.90 (d, J = 8.2 Hz, 1H), 7.78 (dd, J = 7.5, 1.0 Hz, 1H), 7.31 (t, J = 7.9 Hz, 1H), 7.20 (d, J = 3.2 Hz, 1H), 7.00 (d, J = 3.2 Hz, 1H), 5.70 (s, 2H). | 386.02 (M − 1) |
| 32 | 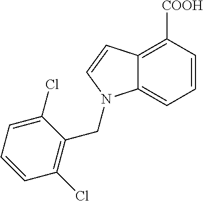 [SMILES: O=C(O)c1cccc2c1ccn2Cc1c(Cl)cccc1Cl] | ¹H NMR (400 MHz, DMSO-d₆) δ 12.71 (s, 1H), 7.90 (d, J = 8.2 Hz, 1H), 7.77 (d, J = 7.4 Hz, 1H), 7.65-7.59 (m, 2H), 7.54-7.45 (m, 1H), 7.29 (t, J = 7.8 Hz, 1H), 7.14 (d, J = 3.3 Hz, 1H), 6.99 (d, J = 3.2 Hz, 1H), 5.63 (s, 2H). | 318.02 (M − 1). |
| 33 | 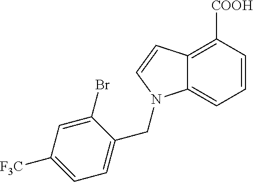 [SMILES: O=C(O)c1cccc2c1ccn2Cc1ccc(C(F)(F)F)cc1Br] | ¹H NMR (400 MHz, DMSO-d₆) δ 12.78 (s, 1H), 8.13-8.09 (m, 1H), 7.79 (dd, J = 7.5, 0.9 Hz, 1H), 7.70-7.63 (m, 3H), 7.23 (t, J = 7.8 Hz, 1H), 7.12 (d, J = 3.1 Hz, 1H), 6.53 (d, J = 8.1 Hz, 1H), 5.66 (s, 2H). | 395.99 (M − 1). |
| 34 | 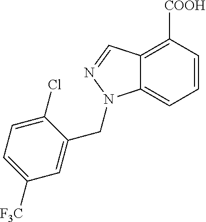 [SMILES: O=C(O)c1cccc2c1cnn2Cc1cc(C(F)(F)F)ccc1Cl] | ¹H NMR (400 MHz, DMSO-d₆) δ 13.23 (s, 1H), 8.46 (d, J = 0.9 Hz, 1H), 8.09 (d, J = 8.4 Hz, 1H), 7.85 (d, J = 7.1 Hz, 1H), 7.80-7.70 (m, 2H), 7.56 (dd, J = 8.4, 7.2 Hz, 1H), 7.38 (d, J = 2.0 Hz, 1H), 5.88 (s, 2H). | 353.04 (M − 1). |
| 35 | 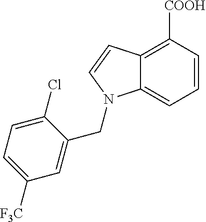 [SMILES: O=C(O)c1cccc2c1ccn2Cc1cc(C(F)(F)F)ccc1Cl] | ¹H NMR (400 MHz, DMSO-d₆) δ 7.83-7.68 (m, 5H), 7.64 (d, J = 3.1 Hz, 1H), 7.24 (t, J = 7.8 Hz, 1H), 7.10 (d, J = 3.1 Hz, 1H), 6.99 (d, J = 2.3 Hz, 1H), 5.69 (s, 2H). | 352.04 (M − 1). |
| 36 | 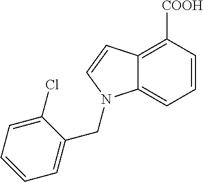 [SMILES: O=C(O)c1cccc2c1ccn2Cc1ccccc1Cl] | ¹H NMR (400 MHz, DMSO-d₆) δ 7.80-7.74 (m, 1H), 7.69 (d, J = 8.2 Hz, 1H), 7.61 (d, J = 3.1 Hz, 1H), 7.53 (dd, J = 8.0, 1.3 Hz, 1H), 7.33 (td, J = 7.7, 1.7 Hz, 1H), 7.28-7.18 (m, 2H), 7.08 (d, J = 3.2 Hz, 1H), 6.64 (dd, J = 7.8, 1.7 Hz, 1H), 5.60 (s, 2H). | 284.06 (M − 1). |
| 37 | 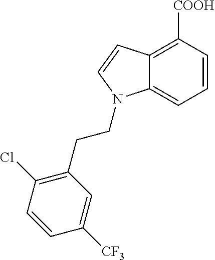 [SMILES: O=C(O)c1cccc2c1ccn2CCc1cc(C(F)(F)F)ccc1Cl] | ¹H NMR (400 MHz, DMSO-d₆) δ 12.68 (s, 1H), 7.89 (s, 1H), 7.77 (t, J = 7.2 Hz, 2H), 7.65 (d, J = 8.1 Hz, 1H), 7.54-7.45 (m, 2H), 7.26 (td, J = 7.9, 2.2 Hz, 1H), 6.99 (d, J = 2.8 Hz, 1H), 4.59 (t, J = 7.3 Hz, 2H), 3.41-3.32 (m, 2H). | 366.06 (M − 1). |

### Example 2. Compound 38

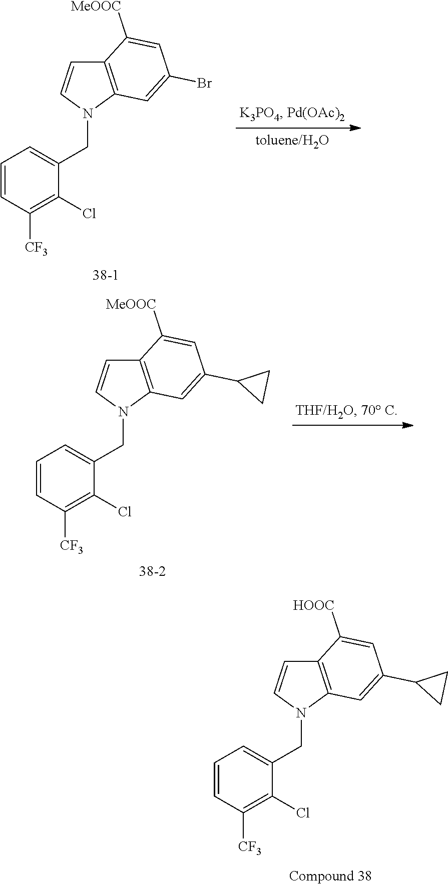 [SMILES: COC(=O)c1cc(Br)cc2c1ccn2Cc1cccc(C(F)(F)F)c1Cl ; COC(=O)c1cc(C2CC2)cc2c1ccn2Cc1cccc(C(F)(F)F)c1Cl ; O=C(O)c1cc(C2CC2)cc2c1ccn2Cc1cccc(C(F)(F)F)c1Cl]

Step 1: Compound 38-1 was prepared in a similar way as described for compound 1-2. To a stirred solution of compound 38-1 (100 mg, 0.22 mmol) in toluene/H₂O (10:1, 5.5 mL) at rt was added K₃PO₄ (166 mg, 0.78 mmol), cyclopropylboronic acid (23.1 mg, 0.268 mmol) and Pd(OAc)₂ (2.8 mg, 0.011 mmol) under N₂. The reaction mixture was stirred at 120° C. overnight. The mixture was extracted with EA and H₂O. The organic layer was dried over Na₂SO₄, filtered, and concentrated in vacuo. The crude product was purified by silica gel chromatography to give 38-2 (46 mg, yield 51.3%). LC-MS, ES⁺ (m/z): 407.09 (M+1).

Step 2: To a stirred solution of compound 38-2 (46 mg, 0.113 mmol) in THF/H₂O (1:1, 2 mL) at rt was added NaOH (23 mg, 0.565 mmol). The resulting solution was stirred at 70° C. overnight. The mixture was acidified to pH 2 with 1 M aq. HCl and extracted with EA and H₂O. The organic layer was dried over Na₂SO₄, filtered, and concentrated in vacuo. The crude product was purified by silica gel chromatography to give Compound 38 (18 mg, yield 40.4%) as a white solid. LC-MS, ES⁺ (m/z): 394.09 (M+1). ¹H NMR (400 MHz, DMSO-d₆) § 12.71 (s, 1H), 7.84-7.77 (m, 1H), 7.55-7.51 (m, 2H), 7.50-7.41 (m, 2H), 7.01 (dd, J=3.2, 0.8 Hz, 1H), 6.66 (d, J=7.7 Hz, 1H), 5.68 (s, 2H), 2.09-2.01 (m, 1H), 0.99-0.92 (m, 2H), 0.72-0.65 (m, 2H).

### Example 3. Compound 39

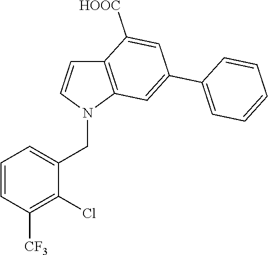 [SMILES: O=C(O)c1cc(-c2ccccc2)cc2c1ccn2Cc1cccc(C(F)(F)F)c1Cl]

Compound 39 was prepared following the same protocol as descried in Compound 38. LC-MS, ES⁺ (m/z): 430.08 (M+1). ¹H NMR (400 MHz, Chloroform-d) δ 8.38 (d, J=1.5 Hz, 1H), 7.71-7.65 (m, 4H), 7.49 (td, J=7.2, 6.5, 1.1 Hz, 2H), 7.42-7.36 (m, 3H), 7.24 (t, J=7.8 Hz, 1H), 6.70-6.64 (m, 1H), 5.63 (s, 2H).

### Example 4. Compound 40

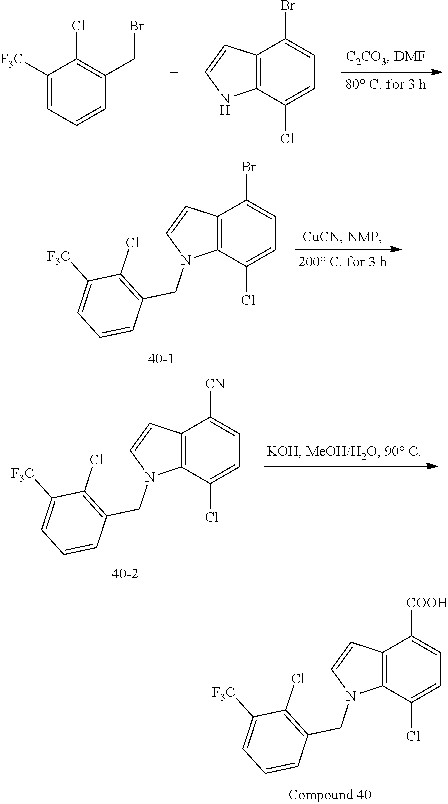 [SMILES: FC(F)(F)c1cccc(CBr)c1Cl ; Clc1ccc(Br)c2cc[nH]c12 ; FC(F)(F)c1cccc(Cn2ccc3c(Br)ccc(Cl)c32)c1Cl ; N#Cc1ccc(Cl)c2c1ccn2Cc1cccc(C(F)(F)F)c1Cl ; O=C(O)c1ccc(Cl)c2c1ccn2Cc1cccc(C(F)(F)F)c1Cl]

Step 1: To a stirred solution of 4-bromo-7-chloro-1H-indole (220 mg, 1.0 mmol) in DMF (5 mL) at rt was added Cs₂CO₃ (650 mg, 2.0 mmol, 2.0 eq), and 1-(bromomethyl)-2-chloro-3-(trifluoromethyl)benzene (270 mg, 1.0 mmol). The reaction mixture was stirred at 80° C. for 3 h. The mixture was extracted with EA and H₂O. The organic layer was dried over Na₂SO₄, filtered, and concentrated in vacuo. The crude product was purified by silica gel chromatography to give 40-1 (290 mg, yield 68.6%). LC-MS, ES⁺ (m/z): 421.89 (M+1).

Step 2: To a stirred solution of 40-1 (170 mg, 0.40 mmol) in NMP (3 mL) at rt was added CuCN (54 mg, 0.61 mmol). The reaction mixture was stirred at 200° C. for 3 h. The mixture was extracted with EA and H₂O. The organic layer was dried over Na₂SO₄, filtered, and concentrated in vacuo. The crude product was purified by silica gel chromatography to give 40-2 (22 mg, yield 14.9%). LC-MS, ES⁺ (m/z): 369.06 (M+1).

Step 3: To a stirred solution of 40-2 (22 mg, 0.06 mmol) in MeOH/H₂O (1:1, 2 mL) at rt was added KOH (33 mg, 0.60 mmol). The resulted solution was stirred at 90° C. overnight. The mixture was acidified with HCl (1N, aq) to pH 2, and extracted with EA and H₂O. The organic layer was dried over Na₂SO₄, filtered, and concentrated in vacuo. The crude product was purified by silica gel chromatography to give Compound 40 (5 mg, yield 21.5%) as a white solid. LC-MS, ES⁺ (m/z): 388.05 (M+1). ¹H NMR (400 MHz, Methanol-d⁴) δ 7.92 (d, J=7.7 Hz, 1H), 7.68 (d, J=7.8 Hz, 1H), 7.52 (d, J=3.3 Hz, 1H), 7.38 (d, J=3.2 Hz, 1H), 7.35 (d, J=7.6 Hz, 1H), 7.25 (t, J=7.9 Hz, 1H), 6.39 (d, J=7.9 Hz, 1H), 5.87 (s, 2H).

### Example 5. Compound 41

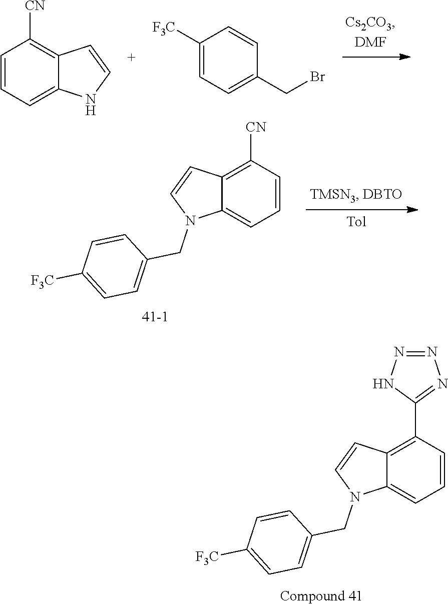

Step 1: 1H-indole-4-carbonitrile (100 mg, 0.703 mmol), 1-(bromomethyl)-4-(trifluoromethyl)benzene (202 mg, 0844 mmol) and Cs₂CO₃ (688 mg, 2.11 mmol) were added to DMF (3 mL) and the resulting mixture was stirred at 80° C. for 2 h. After complete conversion, the reaction mixture was cooled to room temperature, quenched with water, and extracted with EA for three times. The combined organic layer was washed with brine, dried over anhydrous Na₂SO₄, filtered, and concentrated in vacuo. Purification of the residue via column chromatography afforded 41-1 (200 mg, 95% yield) as colorless oil. 1H NMR (400 MHz, Chloroform-d) δ 7.62-7.60 (m, 2H), 7.53 (m, 1H), 7.47 (m, 1H), 7.36 (m, 1H), 7.27-7.19 (m, 3H), 6.85 (m, 1H), 5.47 (s, 2H).

Step 2: Compound 41-1 (45 mg, 0.15 mmol) and TMSN₃ (34.5 mg, 0.3 mmol) were dissolved in toluene (1 mL). Dibutyltin oxide (3.7 mg, 0.015 mmol) was added. The mixture was stirred at 105° C. for 15 h and cooled to room temperature. The reaction mixture was quenched with MeOH, and concentrated in vacuo. The residue was purified by chromatography on silica gel to afford compound 41 (15 mg, 29% yield) as white solid. LC-MS, ES (m/z): 343.92 (M+1). 1H NMR (400 MHz, Methanol-D4) δ 7.66 (m, 1H), 7.60-7.54 (m, 4H), 7.32-7.28 (m, 3H), 7.21 (m, 1H), 5.59 (s, 2H).

The following examples were prepared employing the same protocol as described for Compound 41.

| Compound | Structure | NMR | MS |
|---|---|---|---|
| 42 | 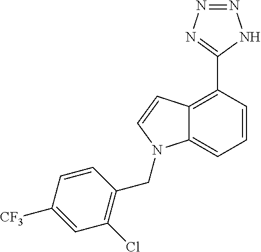 [SMILES: FC(F)(F)c1ccc(Cn2ccc3c(-c4nnn[nH]4)cccc32)c(Cl)c1] | ¹H NMR (400 MHz, Methanol-d₄) δ 7.81 (s, 1H), 7.70 (d, J = 7.4 Hz, 1H), 7.55-7.50 (m, 2H), 7.47 (d, J = 8.1 Hz, 1H), 7.37-7.30 (m, 1H), 7.27 (d, J = 2.9 Hz, 1H), 6.75 (d, J = 8.2 Hz, 1H), 5.69 (s, 2H). | 377.97 (M + 1) |
| 43 | 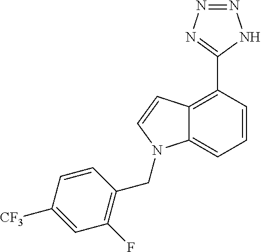 [SMILES: Fc1cc(C(F)(F)F)ccc1Cn1ccc2c(-c3nnn[nH]3)cccc21] | ¹H NMR (400 MHz, Methanol-d₄) δ 7.67 (d, J = 7.4 Hz, 1H), 7.60 (d, J = 8.4 Hz, 1H), 7.57- 7.45 (m, 2H), 7.41-7.28 (m, 2H), 7.25-7.17 (m, 1H), 7.04 (t, J = 7.6 Hz, 1H), 5.63 (s, 2H). | 361.96 (M + 1) |
| 44 | 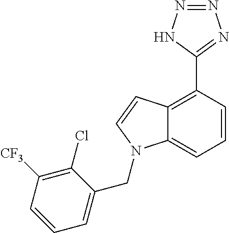 [SMILES: FC(F)(F)c1cccc(Cn2ccc3c(-c4nnn[nH]4)cccc32)c1Cl] | ¹H NMR (400 MHz, DMSO-d₆) δ 7.78 (dd, J = 11.3, 7.6 Hz, 2H), 7.72-7.59 (m, 2H), 7.43 (t, J = 7.9 Hz, 1H), 7.37-7.25 (m, 2H), 6.74 (d, J = 7.8 Hz, 1H), 5.73 (s, 2H). | 377.88 (M + 1) |
| 45 | 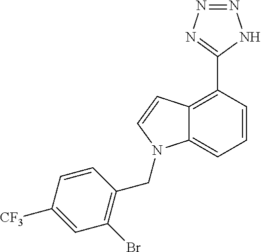 [SMILES: FC(F)(F)c1ccc(Cn2ccc3c(-c4nnn[nH]4)cccc32)c(Br)c1] | ¹H NMR (400 MHz, Methanol-d₄) δ 7.98 (s, 1H), 7.76-7.66 (m, 1H), 7.57-7.46 (m,3H), 7.39-7.30 (m, 1H), 7.27 (d, J = 3.2 Hz, 1H), 6.67 (d, J = 8.4 Hz, 1H), 5.66 (s, 2H). | 422.05 (M + 1) |
| 46 | 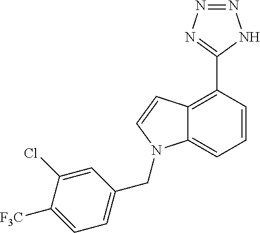 [SMILES: FC(F)(F)c1ccc(Cn2ccc3c(-c4nnn[nH]4)cccc32)cc1Cl] | 1H NMR (400 MHz, Methanol-d4) δ 7.81 (s, 1H), 7.69 (d, J = 7.4 Hz, 1H), 7.57-7.50 (m, 2H), 7.46 (d, J = 8.3 Hz, 1H), 7.37-7.30 (m, 1H), 7.26 (d, J = 3.2 Hz, 1H), 6.78-6.71 (m, 1H), 5.67 (s, 2H). | 377.97 (M + 1) |
| 47 | 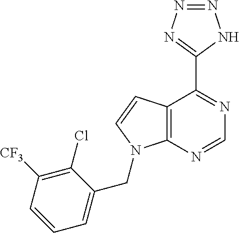 [SMILES: FC(F)(F)c1cccc(Cn2ccc3c(-c4nnn[nH]4)ncnc32)c1Cl] | ¹H NMR (400 MHz, DMSO-d₆) δ 9.01 (s, 1H), 7.95 (d, J = 3.6 Hz, 1H), 7.82 (d, J = 8.0 Hz, 1H), 7.47 (t, J = 7.8 Hz, 1H), 7.29 (d, J = 3.6 Hz, 1H), 7.09 (d, J = 7.7 Hz, 1H), 5.76 (s, 2H). | 379.98 (M + 1) |
| 48 | 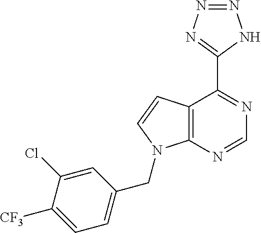 [SMILES: FC(F)(F)c1ccc(Cn2ccc3c(-c4nnn[nH]4)ncnc32)cc1Cl] | ¹H NMR (400 MHz, DMSO-d₆) δ 9.01 (s, 1H), 7.97 (s, 2H), 7.64 (d, J = 8.2 Hz, 1H), 7.30 (d, J = 3.4 Hz, 1H), 7.07-6.96 (m, 1H), 5.75 (s, 2H). | 380.04 (M + 1) |

### Example 6. Compound 49

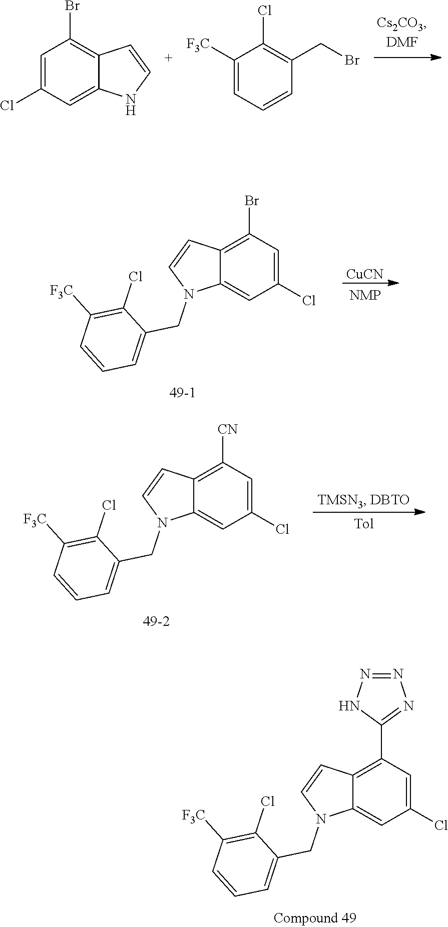

Step 1:4-bromo-6-chloro-1H-indole (400 mg, 1.735 mmol), 1-(bromomethyl)-2-chloro-3-(trifluoromethyl)benzene (480 mg, 1.758 mmol) and Cs₂CO₃ (1696 mg, 5.205 mmol) were added to DMF (8 mL) and the resulting mixture was stirred at 80° C. for 2 h. After complete conversion, the reaction mixture was cooled to room temperature, quenched with water, and extracted with EA for three times. The combined organic layer was washed with brine, dried over anhydrous Na₂SO₄, filtered, and concentrated in vacuo. Purification of the residue via column chromatography afforded 49-1 (626 mg, 85% yield). ¹H NMR (400 MHz, Chloroform-d) δ 7.66-7.64 (m, 1H), 7.34-7.33 (m, 1H), 7.24-7.20 (m, 1H), 7.17-7.15 (m, 2H), 6.65-6.64 (m, 1H), 6.59-6.57 (m, 1H), 5.42 (s, 2H).

Step 2: To a solution of compound 49-1 (100 mg, 0.236 mmol) dissolved in NMP (1 mL) was added CuCN (25 mg, 0.284 mmol) and the mixture was stirred at 180° C. for 3 h. The reaction was cooled to room temperature, quenched with water, and extracted with EA three times. The organic fractions were combined, washed with NaCl, and concentrated in vacuo. The residue was purified by chromatography on silica gel to afford 49-2 (59 mg, 67% yield). ¹H NMR (400 MHz, Chloroform-d) δ 7.69-7.67 (m, 1H), 7.49 (m, 1H), 7.44-7.43 (m, 1H), 7.31-7.30 (m, 1H), 7.28-7.24 (m, 1H), 6.83 (m, 1H), 6.63-6.61 (m, 1H), 5.49 (s, 2H).

Step 3: Compound 49-2 (46 mg, 0.124 mmol) and TMSN₃ (29 mg, 0.249 mmol) were dissolved in toluene (1 mL). Dibutyltin oxide (3 mg, 0.0124 mmol) was added. The mixture was heated at 110° C. for 15 h and cooled to room temperature. The reaction mixture was quenched with MeOH, and concentrated in vacuo. The residue was purified by chromatography on silica gel to afford Compound 49 (12 mg, 23% yield) as white solid. LC-MS, ES (m/z): 412.01 (M+1). ¹H NMR (400 MHz, Di methyl Sulfoxide-D6) δ 7.94-7.92 (m, 1H), 7.81-7.78 (m, 2H), 7.70-7.69 (m, 1H), 7.45-7.41 (m, 1H), 7.29-7.28 (m, 1H), 6.70-6.68 (m, 1H), 5.73 (s, 2H).

The following examples were prepared employing the same protocol as described for Compound 49.

| Compound | Structure | NMR | MS |
|---|---|---|---|
| 50 | 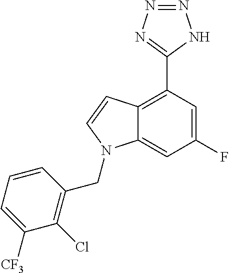 [SMILES: Fc1cc(-c2nnn[nH]2)c2ccn(Cc3cccc(C(F)(F)F)c3Cl)c2c1] | ¹H NMR (400 MHz, DMSO-d₆) δ 7.79 (d, J = 8.2 Hz, 1H), 7.70 (dd, J = 9.7, 1.9 Hz, 1H), 7.67- 7.60 (m, 2H), 7.44 (t, J = 7.9 Hz, 1H), 7.27 (d, J = 2.7 Hz, 1H), 6.73 (d, J = 7.8 Hz, 1H), 5.70 (s, 2H). | 396.03 (M + 1) |
| 51 | 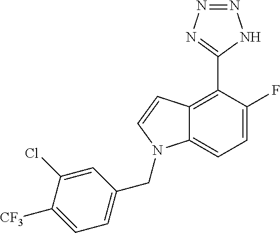 [SMILES: Fc1ccc2c(ccn2Cc2ccc(C(F)(F)F)c(Cl)c2)c1-c1nnn[nH]1] | ¹H NMR (400 MHz, DMSO-d₆) δ 7.97 (d, J = 1.9 Hz, 1H), 7.73 (dd, J = 15.1, 3.6 Hz, 2H), 7.63 (dd, J = 8.0, 1.8 Hz, 1H), 7.25 (dd, J = 11.1, 9.0 Hz, 1H), 7.17-7.13 (m, 1H), 6.70 (d, J = 8.1 Hz, 1H), 5.73 (s, 2H). | 396.10 (M + 1) |
| 52 | 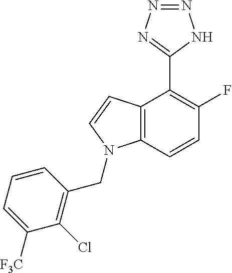 [SMILES: Fc1ccc2c(ccn2Cc2cccc(C(F)(F)F)c2Cl)c1-c1nnn[nH]1] | 1H NMR (400 MHz, DMSO- d6) δ 7.80 (dd, J = 7.9, 1.6 Hz, 1H), 7.75 (dd, J = 8.7, 3.8 Hz, 2H), 7.44 (t, J = 7.9 Hz, 1H), 7.25 (dd, J = 11.1, 9.0 Hz, 1H), 7.15 (d, J = 3.1 Hz, 1H), 6.75 (d, J = 7.5 Hz, 1H), 5.74 (s, 2H). | 396.06 (M + 1) |

### Example 7. Compound 53

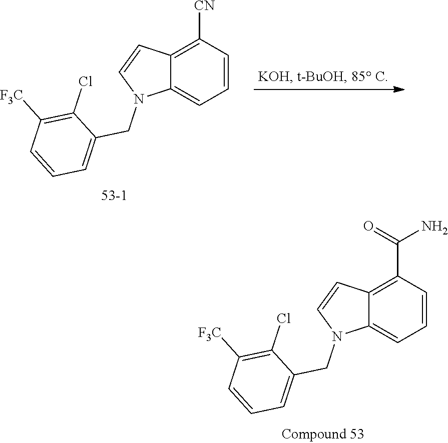 [SMILES: N#Cc1cccc2c1ccn2Cc1cccc(C(F)(F)F)c1Cl ; NC(=O)c1cccc2c1ccn2Cc1cccc(C(F)(F)F)c1Cl]

1-(2-chloro-3-(trifluoromethyl)benzyl)-1H-indole-4-carbonitrile (50 mg, 0.150 mmol) and KOH (142.8 mg, 2.545 mmol) were dissolved in t-BuOH (2 mL) and the resulting solution was stirred at 85° C. for 16 h. After complete conversion, the reaction mixture was cooled to room temperature, neutralized with HCl, and extracted with EA for three times. The combined organic layer was washed with brine, dried over anhydrous Na₂SO₄, filtered, and concentrated in vacuo. The residue was purified by prep-TLC to give Compound 53 (5.86 mg, 11% yield) as white solid. LC-MS, ES (m/z): 352.97 (M+1). ¹H NMR (400 MHz, Chloroform-d) δ 7.63 (d, J=7.9 Hz, 1H), 7.57 (d, J=7.3 Hz, 1H), 7.36 (d, J=8.2 Hz, 1H), 7.29 (d, J=3.3 Hz, 1H), 7.20 (dd, J=24.3, 7.9 Hz, 2H), 7.11 (d, J=3.2 Hz, 1H), 6.56 (d, J=7.8 Hz, 1H), 5.53 (s, 2H).

### Example 8. Compound 54

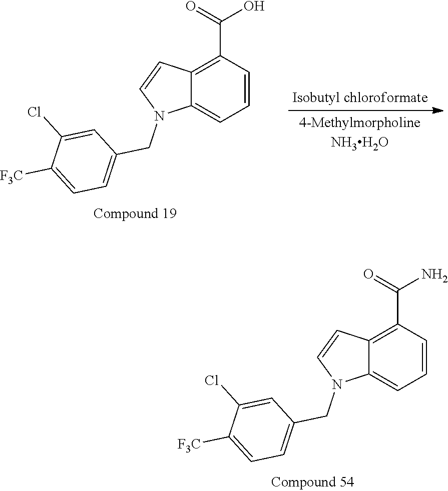 [SMILES: O=C(O)c1cccc2c1ccn2Cc1ccc(C(F)(F)F)c(Cl)c1 ; NC(=O)c1cccc2c1ccn2Cc1ccc(C(F)(F)F)c(Cl)c1]

A solution of 1-(3-chloro-4-(trifluoromethyl)benzyl)-1H-indole-4-carboxylic acid (Compound 19) (20 mg, 0.057 mmol) and 4-methylmorpholine (8.6 mg, 0.085 mmol) in THF (2 mL) was cooled to −15° C. and isobutyl chloroformate (11.6 mg, 0.085 mmol) was added. The resulting solution was stirred at −15° C. for 1 h. NH₃·H₂O (29%, 2 mL) was added and the mixture was warmed to room temperature over 1 h. The reaction was quenched with water and extracted with EA for three times. The combined organic layer was washed with brine, dried over anhydrous Na₂SO₄, filtered, and concentrated in vacuo. The residue was purified by prep-TLC to give Compound 54 (3.46 mg, 17% yield) as white solid. LC-MS, ES (m/z): 352.94 (M+1). ¹H NMR (400 MHz, Chloroform-d) δ 7.70 (d, J=1.7 Hz, 1H), 7.57 (dd, J=7.3, 0.9 Hz, 1H), 7.37-7.31 (m, 2H), 7.29 (d, J=3.2 Hz, 1H), 7.24 (t, J=7.8 Hz, 1H), 7.10 (dd, J=3.3, 0.8 Hz, 1H), 6.56 (d, J=8.1 Hz, 1H), 5.51 (s, 2H).

### Example 9. Compound 55

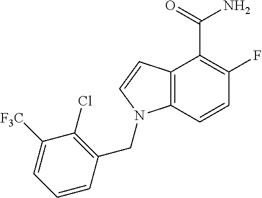 [SMILES: NC(=O)c1c(F)ccc2c1ccn2Cc1cccc(C(F)(F)F)c1Cl]

Compound 55 was prepared following the same protocol as described in Compound 54. LC-MS, ES (m/z): 371.10 (M+1). ¹H NMR (400 MHz, Chloroform-d) δ 7.63 (d, J=7.8 Hz, 1H), 7.40 (s, 1H), 7.30 (s, 1H), 7.24 (s, 1H), 7.18 (t, J=7.9 Hz, 1H), 6.98 (t, J=10.5 Hz, 1H), 6.54 (d, J=7.8 Hz, 1H), 5.53-5.46 (m, 2H).

### Example 10. Provided Technologies can Reduce MRGPRX4 Activation

Among other things, provided technologies can reduce MRGPRX4 activation. Various technologies can be utilized in accordance with the present disclosure to access activities of provided compounds. An example is described below.

HEK293T cells stably expressing human MRGPRX4 were cultured in DMEM medium supplements with 10% FBS, 100 U/mL penicillin-streptomycin, 20 mM HEPES and 1 μg/mL puromycin at 37° C. with 5% (v/v) CO₂. One day before test, the cells were detached using TrypLE™ Express and counted using cell counter. In a 384-well plate cells were seeded at a density of 20,000 cells/well in 20 μL/well planting medium (DMEM medium supplements with 5% FBS, 100 U/mL penicillin-streptomycin and 20 mM HEPES), and incubated overnight at 37° C. with 5% (v/v) CO₂. On the day of experiments, the dye solution, prepared following the manufacture's instruction of the Screen Quest™ Fluo-8 No Wash Calcium Assay Kit, was added to each well (20 μL/well) of the cell plate. The cell plate was incubated at 25° C. for 1 hour. Test compounds in different doses were transferred to the cell plate by FLIPR and the plate was kept in the dark at 25° C. for 30 minutes. The agonist solution, 0.2 μM of deoxycholic acid-3-phosphoric acid in assay buffer, was added to each well of the plate. The plate was read for 160 sec with 1 sec interval to obtain data of antagonist mode in FLIPR. For various compounds, IC₅₀ values were calculated by fitting % inhibition against log of compound concentrations (top conc. 30 μM, ten 3-fold serial dilutions) with Hill equation using XLfit. MRGPRX4 antagonist activities of certain compounds tested in this assay were listed in Table 1 as examples. Those skilled in the art appreciate that IC₅₀ of various compounds can be assessed in accordance with the present disclosure. In some embodiments, inhibition of various compounds was assessed at certain concentrations, e.g., about 0.5 μM, about 2 μM, etc; certain data were presented in the Table below as examples.

TABLE 1
Certain compounds and data as examples (A =
IC₅₀ < 0.5 μM; B = 0.5 μM <= IC₅₀ <= 5 μM; C = IC₅₀ > 5 μM).

| | % inhibition @ | % inhibition @ | |
|---|---|---|---|
| Compound | 2 μM | 500 nM | IC50 |
| 1 | n/a | 78% | A |
| 2 | n/a | 98% | A |
| 3 | 34% | n/a | B |
| 4 | 91% | n/a | A |
| 5 | 40% | n/a | C |
| 6 | 18% | n/a | n/a |
| 7 | n/a | 20% | n/a |
| 8 | n/a | 64% | A |
| 9 | n/a | −1.6% | n/a |
| 10 | n/a | 90% | A |
| 11 | n/a | −1.2% | n/a |
| 12 | 100.7% | n/a | A |
| 13 | n/a | 27% | n/a |
| 14 | n/a | 12% | n/a |
| 15 | n/a | 5.3% | n/a |
| 16 | 94% | n/a | A |
| 17 | 3.6% | n/a | n/a |
| 18 | 7.2% | n/a | n/a |
| 19 | 99.7% | n/a | A |
| 20 | 97% | n/a | B |
| 21 | 52% | n/a | n/a |
| 22 | 2.4% | n/a | n/a |
| 23 | 29% | n/a | n/a |
| 24 | n/a | 20% | n/a |
| 25 | n/a | n/a | C |
| 26 | 49% | n/a | n/a |
| 27 | 72% | n/a | n/a |
| 28 | n/a | 86% | A |
| 29 | n/a | 76% | A |
| 30 | n/a | 97% | A |
| 31 | −6.2% | n/a | n/a |
| 32 | −13% | n/a | n/a |
| 33 | 98% | n/a | B |
| 34 | −2.4% | n/a | n/a |
| 35 | 5.1% | n/a | n/a |
| 36 | 0.4% | n/a | n/a |
| 37 | 5.5% | n/a | n/a |
| 38 | n/a | 44% | n/a |
| 39 | n/a | 1.6% | n/a |
| 40 | n/a | 4.9% | n/a |
| 41 | 81% | n/a | n/a |
| 42 | 100% | n/a | A |
| 43 | 96% | n/a | A |
| 44 | 65% | n/a | B |
| 45 | 100% | n/a | A |
| 46 | 99.9% | n/a | A |
| 47 | n/a | 13% | n/a |
| 48 | n/a | 4.3% | n/a |
| 49 | n/a | −11% | n/a |
| 50 | n/a | −11.2% | n/a |
| 51 | n/a | 99.9% | A |
| 52 | n/a | 45% | A |
| 53 | n/a | −7.6% | n/a |
| 54 | n/a | 11% | n/a |
| 55 | n/a | 36% | n/a |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. A compound has the structure of formula I: 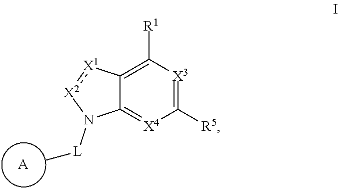
or a salt thereof, wherein: 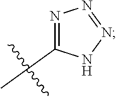 [SMILES: *c1nnn[nH]1]
R¹ is an isostere of —C(O)OH, or —C(O)OR¹¹, —C(O)N(R¹²)SO₂R¹³, —C(O)NR¹⁴R¹⁵, or
—X¹═X²— is —X¹═X²— and X¹ and X² is independently —N═, —CR²═, or optionally substituted —CH═; or —X¹ ═X²— is optionally substituted (CH₂)₂—;
R² is optionally substituted C₁₋₆ alkyl;
X³ is —N═, or —CR³═;
X⁴ is —N═, or —CR⁴═;
each of R³, R⁴ and R⁵ is independently R′, OR′, halogen, —CN, —NO₂, or —N(R′)₂;
each of R¹¹, R¹², R¹³, R¹⁴ and R¹⁵ is independently R′;
L is optionally substituted —(CH₂)ₘ—;
m is 1 or 2;
Ring A is an optionally substituted ring selected from a 6-10 membered aryl ring and a 5-10 membered heteroaryl ring having 1-6 heteroatoms, or Ring A is 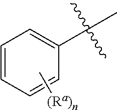
wherein each R^{a} is independently —R′, —OR′, halogen, —CN, —NO₂, or —N(R′)₂ and n is 1-5;
each R′ is independently R, —OR, —C(O)R, —C(O)OR, or —S(O)₂R;
each R is independently hydrogen or an optionally substituted group selected from C₁-C₆ aliphatic, C₁-C₆ heteroaliphatic having 1-3 heteroatoms, 3-10 membered cycloaliphatic, 3-10 membered heterocyclyl having 1-4 heteroatoms, 6-10 membered aryl, 5-10 membered heteroaryl having 1-6 heteroatoms, 6-10 membered aryl-C₁-C₆ aliphatic, and 5-10 membered heteroaryl having 1-6 heteroatoms-C₁-C₆ aliphatic; or
two R groups on the same atom are optionally and independently taken together with the atom to form an optionally substituted 3-10 membered ring having, in addition to the atom, 0-4 heteroatoms; or
two R groups on two atoms are optionally and independently taken together with their intervening atoms to form an optionally substituted 3-10 membered ring having, in addition to the intervening atoms, 0-4 heteroatoms.

2. The compound of claim 1, wherein the compound has a structure of: 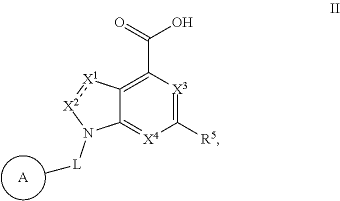
or a salt thereof.

3. The compound of claim 1, wherein the compound has a structure of: 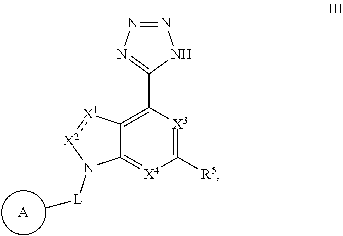
or a salt thereof.

4. The compound of claim 1, wherein the compound has a structure of: 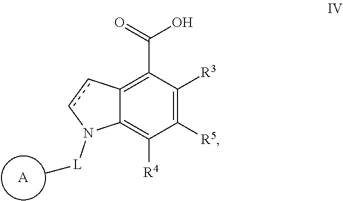
or a salt thereof.

5. The compound of claim 1, wherein the compound has a structure of: 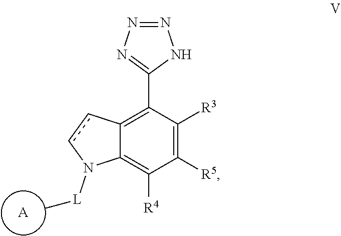
or a salt thereof.

6. A compound, wherein the compound has a structure selected from 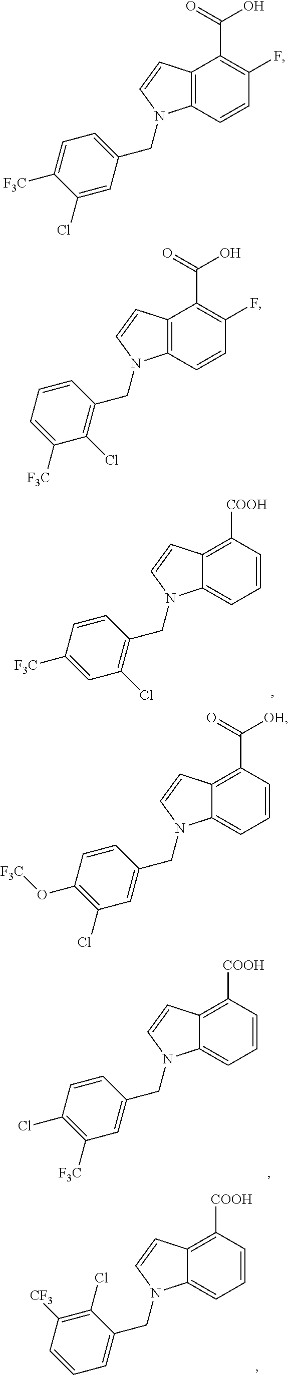 [SMILES: O=C(O)c1c(F)ccc2c1ccn2Cc1ccc(C(F)(F)F)c(Cl)c1 ; O=C(O)c1c(F)ccc2c1ccn2Cc1cccc(C(F)(F)F)c1Cl ; O=C(O)c1cccc2c1ccn2Cc1ccc(C(F)(F)F)cc1Cl ; O=C(O)c1cccc2c1ccn2Cc1ccc(OC(F)(F)F)c(Cl)c1 ; O=C(O)c1cccc2c1ccn2Cc1ccc(Cl)c(C(F)(F)F)c1 ; O=C(O)c1cccc2c1ccn2Cc1cccc(C(F)(F)F)c1Cl] 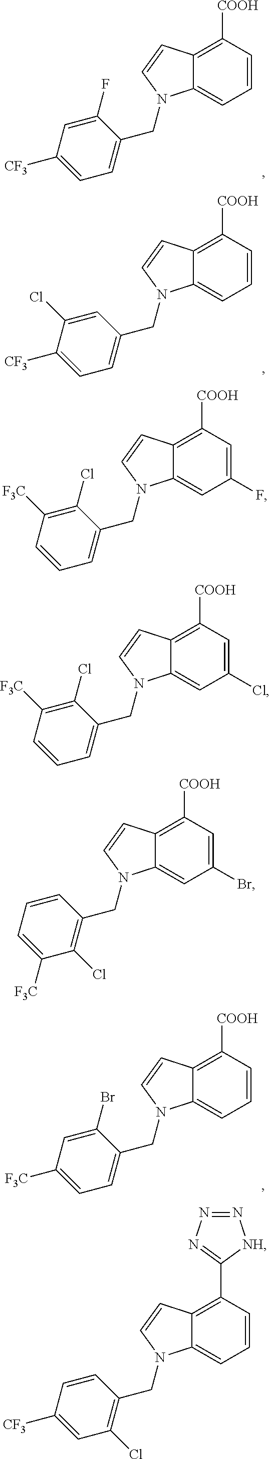 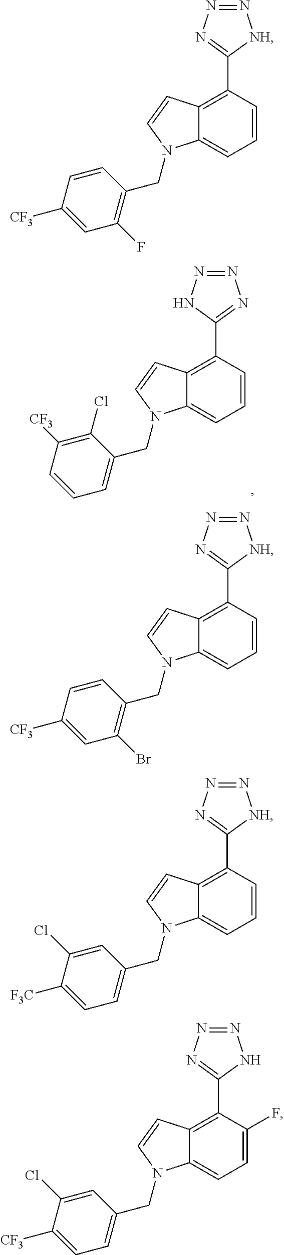 [SMILES: Fc1cc(C(F)(F)F)ccc1Cn1ccc2c(-c3nnn[nH]3)cccc21 ; FC(F)(F)c1cccc(Cn2ccc3c(-c4nnn[nH]4)cccc32)c1Cl ; FC(F)(F)c1ccc(Cn2ccc3c(-c4nnn[nH]4)cccc32)c(Br)c1 ; FC(F)(F)c1ccc(Cn2ccc3c(-c4nnn[nH]4)cccc32)cc1Cl ; Fc1ccc2c(ccn2Cc2ccc(C(F)(F)F)c(Cl)c2)c1-c1nnn[nH]1] 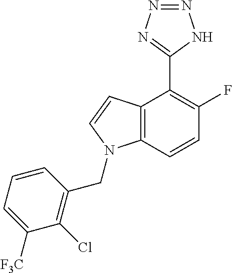 [SMILES: Fc1ccc2c(ccn2Cc2cccc(C(F)(F)F)c2Cl)c1-c1nnn[nH]1]
or a salt thereof.

7. A pharmaceutical composition comprising a compound of any one of the preceding claims and a pharmaceutically acceptable carrier.

8. A method for treating or preventing a MRGPRX4-associated condition, disorder or disease, comprising delivering or administering to a subject suffering therefrom an effective amount of the compound of any one of claims 1-6 or the pharmaceutical composition of claim 7.

9. The method of claim 8, wherein the condition, disorder or disease is or comprises chronic itch, cholestatic pruritus, contact dermatitis, allergic blepharitis, anemia, atopic dermatitis, bullous pemphigoid, candidiasis, chicken pox, cholestasis, end-stage renal failure, hemodialysis, contact dermatitis, dermatitis herpetiformis, diabetes, drug allergy, dry skin, dyshidrotic dermatitis, ectopic eczema, eczema, erythrasma, folliculitis, fungal skin infection, hemorrhoids, herpes, HIV infection, Hodgkin's disease, hyperthyroidism, iron deficiency anemia, kidney disease, leukemia, liver disease, lymphoma, malignancy, multiple myeloma, neurodermatitis, onchocerciasis, Paget's disease, pediculosis, polycythemia rubra vera, pruritus ani, pseudorabies, psoriasis, rectal prolapse, scabies, schistosomiasis, scleroderma, severe stress, stasis dermatitis, swimmer's itch, thyroid disease, tinea cruris, uremic pruritus, or urticaria.

10. The method of claim 9, wherein the liver condition, disorder or disease is intrahepatic cholestasis of pregnancy (ICP), estrogen-, progesterone- or testosterone-induced cholestasis, toxin- or other drug induced hepatocellular cholestasis, benign recurrent intrahepatic cholestasis (BRIC), progressive familial intrahepatic cholestasis (PFIC), chronic viral hepatitis C, chronic hepatitis B, alcoholic or nonalcoholic fatty liver disease (NAFLD), nonalcoholic steatohepatitis (NASH), primary biliary cholangitis (PBC), primary sclerosing cholangitis (PSC), secondary sclerosing cholangitis (SSC), sarcoidosis, ABCB4 deficiency, alagille syndrome, drug-induce small duct cholangiopathies, gallstone disease, IgG4-associated cholangitis, biliary atresia, cholangiocellular carcinoma, benign bile duct adenoma, or other obstructive cholestasis.
